(19) United States
(12) Patent Application Publication
(10) Pub. No.: US 2008/0096907 A1
(43) Pub. Date: Apr. 24, 2008

(54) Aryl, Pyrimidyl Compounds, Pharmaceutical Compositions Comprising them, Their Use as Antimicrobial Agents

(75) Inventors: Helene Munier-Lehmann, Meudon (FR); Dominique Douguet, Le Rouret (FR); Gilles Labesse, Montpellier (FR); Sylvie Pochet, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignees: INSTITUT PASTEUR, Paris (FR); INSTITUT NAT. DE LA SANTE DE LA RECHERCHE MED., Paris (FR); CENTRE NATIONAL DE LA RECHERCHE SCIENTIFIQUE, Paris (FR)

(21) Appl. No.: 11/718,764

(22) PCT Filed: Nov. 4, 2005
(86) PCT No.: PCT/EP05/12346

§ 371 Date: Sep. 4, 2007

(30) Foreign Application Priority Data
Nov. 5, 2004 (EP) 04292629.5

(51) Int. Cl. A61K 31/513; A61P 31/06; C07D 239/545; C07D 239/553

(52) U.S. Cl. 514/274; 544/312; 544/313

(57) ABSTRACT

Substituted aryl pyrimidyl compounds responding to formula (I) and their use for the preparation of a medicament for the prevention and/or treatment of a pathology caused by a mycobacteria.

The present invention relates to certain substituted aryl pyrimidyl compounds and to a process for their synthesis. It also relates to pharmaceutical compositions comprising them and to their use as antimicrobial agents, especially for the prevention or treatment of pathologies in relationship with a mycobacteria.

The invention relates in particular to the use of such molecules for the prevention or treatment of tuberculosis and other diseases caused by a mycobacteria.

The incidence of tuberculosis has been increasing during the last twenty years and it is now the first cause of mortality among infectious diseases in the world, killing more than two million people a year. Mycobacterium tuberculosis (M. tuberculosis) is the principal microbial agent involved for humans. Tuberculosis is primarily transmitted via airborne aerosoled secretions. A peculiar aspect of its pathogenicity comes from the fact that it can remain quiescent and become active decades later. One of the most significant risk factor for developing tuberculosis is human immunodeficiency virus (HIV) infection. The current treatment of active tuberculosis includes four drugs (isoniazid, rifampicin, pyrazinamide and ethambutol) for at least six months. A significant proportion of patients do not complete the therapy, especially in developing countries, and this has led to the appearance of resistant strains of M. tuberculosis.

Consequently, there is a need for new molecules which are efficient against M. tuberculosis.

In this context thymidine monophosphate kinase (TMPK), one essential enzyme of nucleotide metabolism is an interesting target.

TMPK (E.C.2.7.4.9, ATP:TMP phosphotransferase) belongs to a large superfamily of nucleoside monophosphate kinases (NMPK). It catalyses the phosphorylation of thymidine monophosphate (TMP) to thymidine diphosphate (TDP) utilizing ATP as its preferred phosphoryl donor. It lies at the junction of the de novo and salvage pathways of thymidine triphosphate (TTP) metabolism and is the last specific enzyme for its synthesis. These characteristics make TMPK a good target for the design of new antibiotic drugs.

Purine and pyrimidine nucleoside analogues acting on the TMPK of M. tuberculosis have been disclosed in S. Pochet et al., Chem. Bio. Chem. 2003, 4, 742-747.

However there is always a need for molecules with a stronger biological activity, a better specificity, an improved bioavailability, and molecules which would be easier to synthesize, so that their production on industrial scale can be envisioned.

An object of the instant invention is the molecules responding to formula (I): 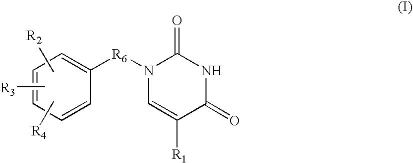
wherein:
R₁ is selected from the group consisting of: CH₃, —CF₃, a halogen atom, —NH₂, —COOH, —CONH₂,
R₂, R₃, R₄, identical or different, are selected from the group consisting of:
   H, a halogen atom,
   C₁-C₈ alkyl, C₂-C₈ alkenyl, C₂-C₈ alkynyl, wherein the alkyl, alkenyl or alkynyl chain may be interrupted by a heteroatom bridge, said heteroatom being preferably selected from: N, S, O, Se
   —OH, —NH₂, —CHO, —COOH, —SO₄H, —CONH₂, —CN, —COOR₅, —COR₅, —OR₅,
   substituted C₁-C₈ alkyl, substituted C₂-C₈ alkenyl, or substituted C₂-C₈ alkynyl wherein the substituent is selected from the group consisting of: —OH, —NH₂, —CHO, —COOH, —SO₄H, —CONH₂, —CN, —COOR₅, —COR₅, —OR₅, a halogen atom, wherein the alkyl, alkenyl or alkynyl chain may be interrupted by a heteroatom bridge, said heteroatom being preferably selected from: N, S, O, Se;
R₅ is selected from the group consisting of C₁-C₆ alkyl;
R₆ is selected among: C₁-C₄ alkyl, C₂-C₄ alkenyl, carbonyl (═C═O), —(CF₂)ₙ
n is an integer selected from 1, 2, 3, and their pharmaceutically acceptable salts.

Alkyl is a linear, branched or cyclic hydrogeno carbon radical.

Alkenyl is a linear, branched or cyclic hydrocarbyl radical comprising at least one double bond.

Alkynyl is a linear, branched or cyclic hydrocarbyl radical comprising at least one triple bond.

Halogen is selected from the group consisting of Cl, F, Br, I.

When the alkyl, alkenyl or alkynyl chain is interrupted by a heteroatom this heteroatom may be divalent or trivalent. In this last case, the heteroatom may be substituted by an alkyl, alkenyl or alkynyl group, which itself may possibly be substituted with one of the functions: —OH, —NH₂, —CHO, —COOH, —SO₄H, —CONH₂, —CN, —COOR₅, —COR₅, —OR₅ or a halogen atom.

Preferentially, the molecule responding to formula (I) satisfies one or more of the following conditions.
R₆ is —CH₂—;
R₁ is selected from the group consisting of: —CH₃, —Br, —Cl;
at least one group among R₂, R₃, R₄ is H.

More preferentially, R₂═R₃═H.

Advantageously R₄ is in the para position on the phenyl ring.

In a preferred manner, R₄ is selected from the group consisting of substituted C₁-C₆ alkyl or substituted C₂-C₆ alkenyl, wherein the substituent is —COOH, possibly comprising a heteroatom bridge, said heteroatom being selected from: N, S, O, Se.

Even more preferentially R₄ is selected from the group consisting of C₂-C₄ alkyl substituted by one —COOH, or a C₁-C₈ alkyl interrupted by a sulphur bridge.

Advantageously, R₄ is 4-yl-n-butyric acid

The favourite molecules are described by their chemical formula here-under:

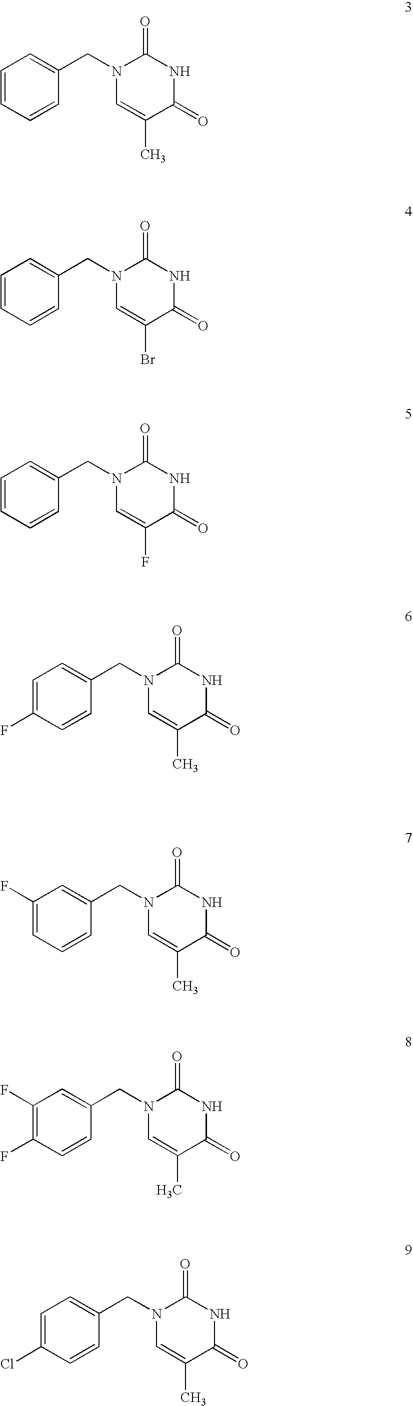

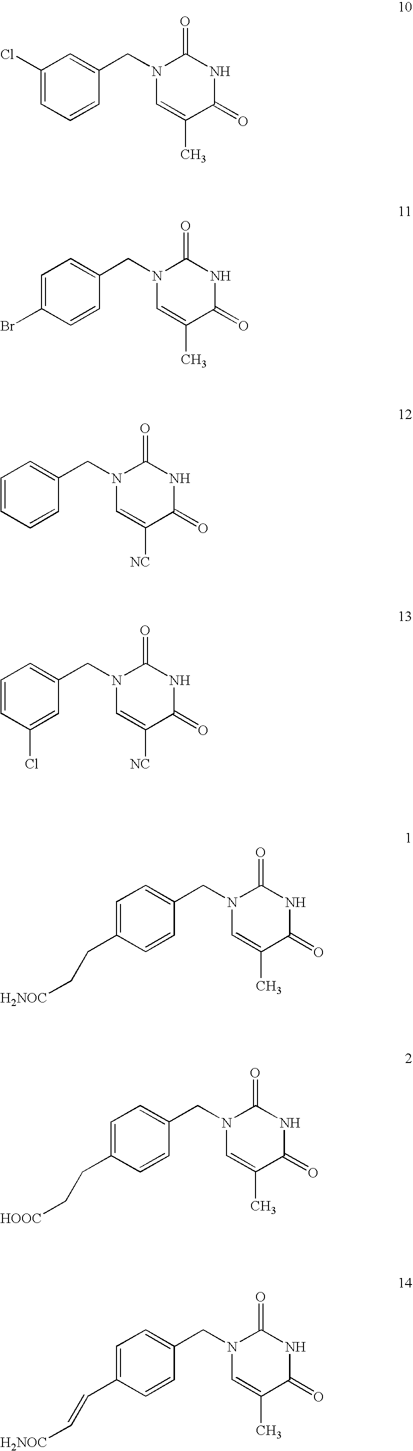

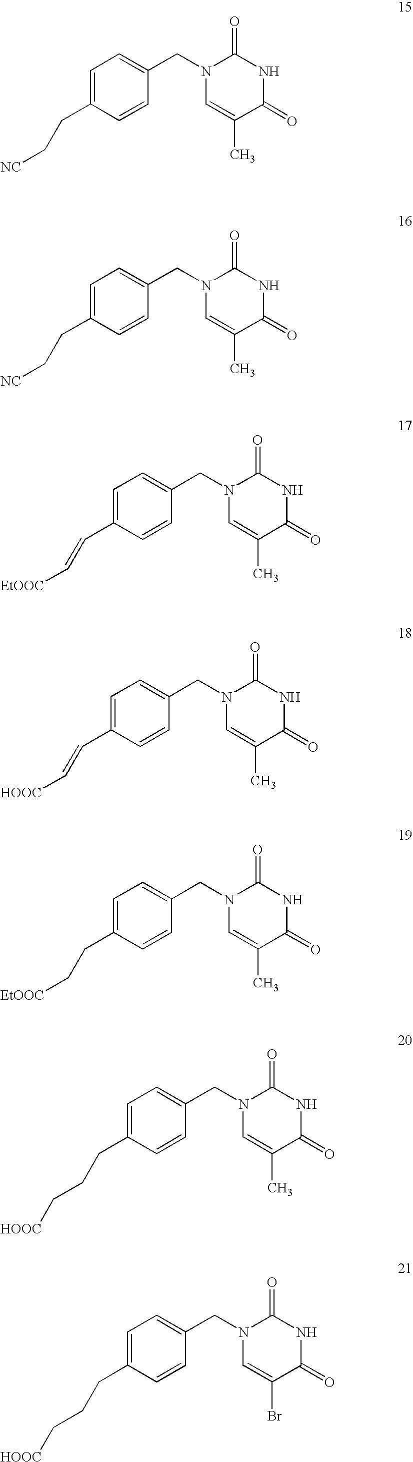

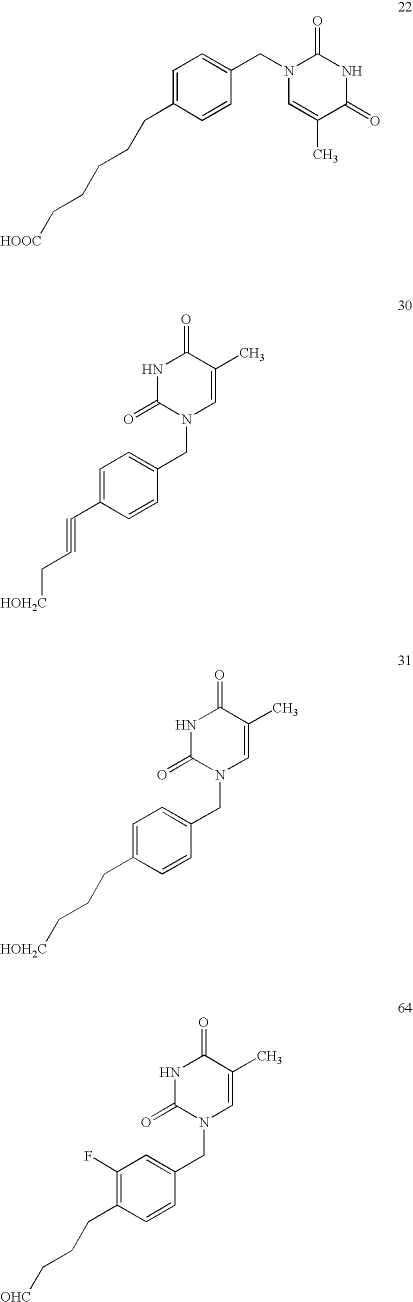

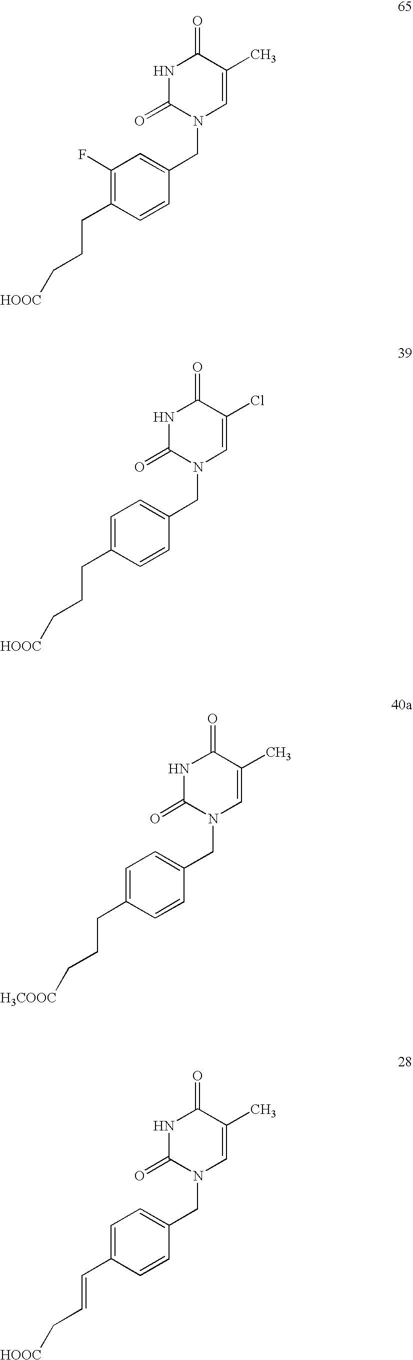

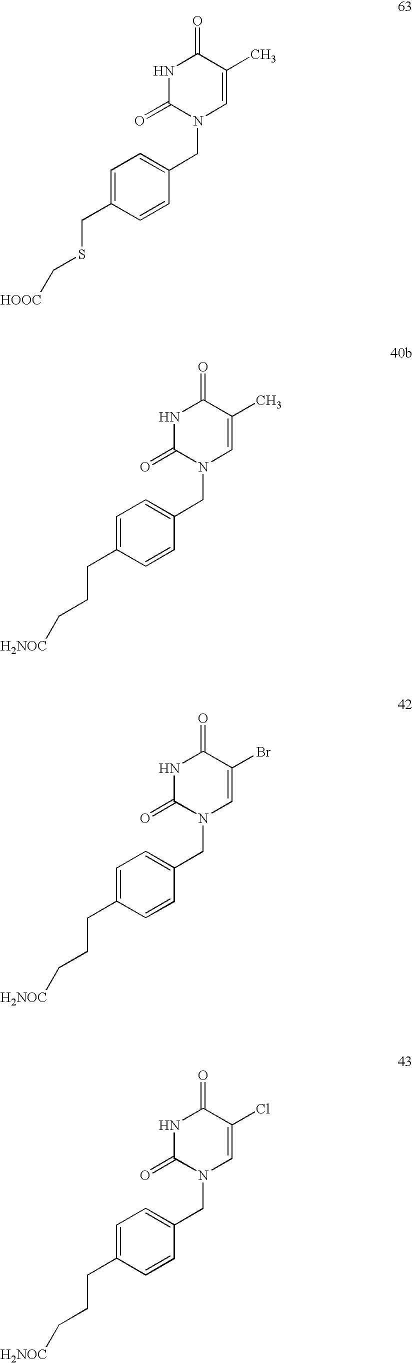

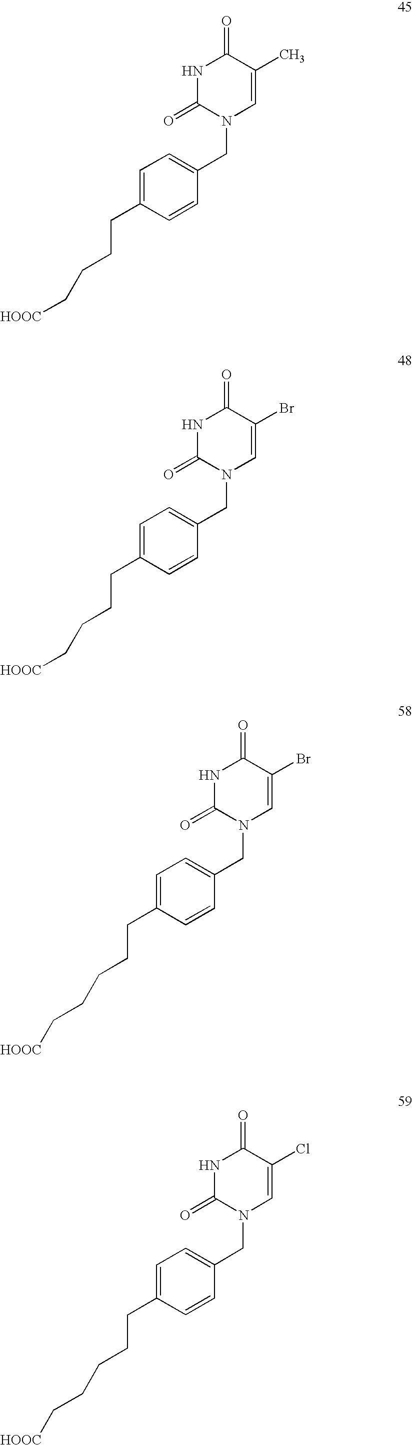

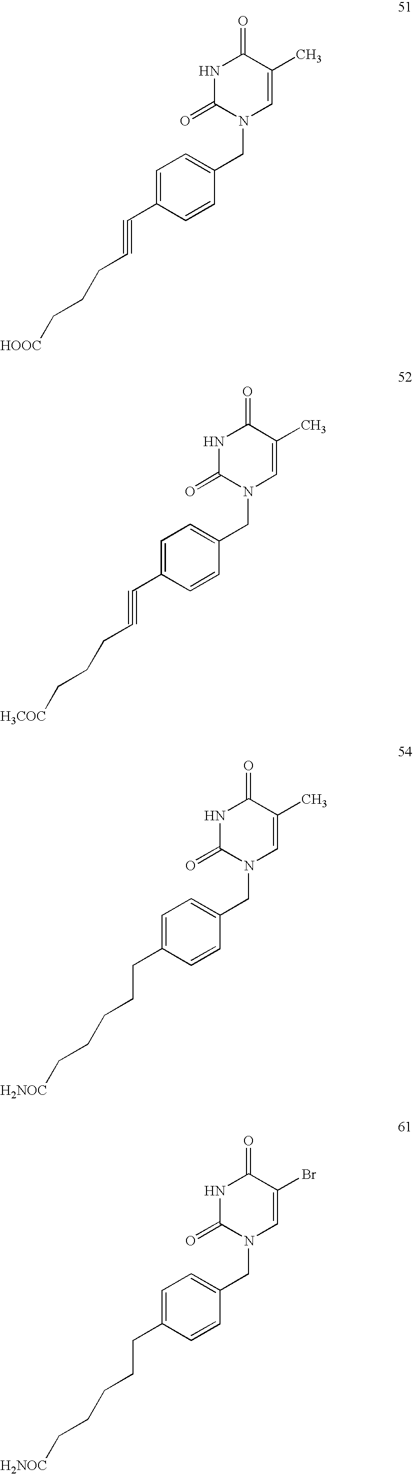

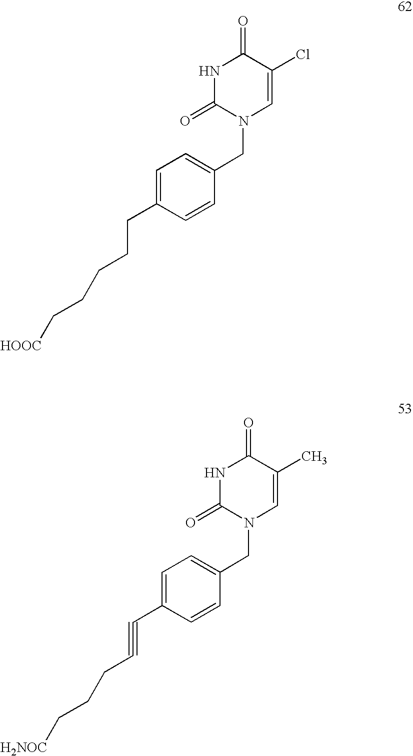

The above described molecules have demonstrated their capacity to inhibit M. tuberculosis TMPK and consequently they can be used for the preparation of a medicament for the prevention and/or treatment of tuberculosis. It must be said that these molecules have a capacity to inhibit M. tuberculosis TMPK in vitro which varies in Ki value, according to which molecule is concerned. However, the inhibitory activity exists and permits to have good hopes of an in vivo inhibition of M. tuberculosis TMPK. Preferred molecules are the ones whose Ki is inferior or equal to 40 μM, and even more preferentially inferior or equal to 30 μM. The molecules 20, 21, 22, 39, 61, 63 and 64 depicted here-above are the favourite molecules for their action as inhibitors of M. tuberculosis TMPK.

The molecules of the invention can also be used as an inhibitor of a mycobacteria TMPK, especially M. tuberculosis TMPK in vitro, for biological tests for example.

Moreover, these molecules can also be used for the preparation of a medicament for the prevention or treatment of other pathologies caused by a mycobacteria, among which: leprosy (M. leprae).

The compounds of the instant invention have the advantage of having a lower affinity for human TMPK than for M. tuberculosis TMPK. Consequently, side effects of a drug based on these compounds administered at therapeutic dosage would be limited.

The instant invention encompasses pharmaceutical compositions comprising at least one compound of formula (I) in a pharmaceutically acceptable carrier. The routes of administration include the oral, buccal, intranasal, ocular, intraveneous, intramuscular, transdermal, parenteral and rectal routes. Preferentially when the pathology to be treated is tuberculosis, the pharmaceutical composition of the invention is administered by oral or intranasal route as tablets, pills, dragees, capsules, gels, suspensions, syrups. It may be distributed in an aerosol or as solution for inhalation or any other form which allows easy volatilization for rapid administration to the lung.

The dosage and posology of the pharmaceutical composition is adapted to the weight, age and condition of the patient and to the inhibitory action of the molecule. The daily dose of active principle is comprised between 0,1 and 500 mg/kg.

Pharmaceutically acceptable salts encompass salts of acid functions of (I) with an organic or an inorganic base and salts of an amine function of (I) with an organic or mineral acid.

Among addition salts with acids are included: acetic, oxalic, succinic, fumaric, gluconic, malic, ascorbic, benzoic, hydrochloric, phosphoric, hydrobromic, sulphuric, sulfinic, formic, toluene sulfonic, methane sulfonic, nitric, benzoic, citric, tartaric, maleic, hydroiodic salts.

Among addition salts with bases are included: sodium, potassium and lithium salts; calcium, barium and magnesium salts; ammonium, ferrous, ferric, zinc, manganese, aluminium, magnesium salts; trimethylamine, triethylamine, tri(n-propyl)amine, dicyclohexylamine, triethanolamine, arginine, lysine, histidine, ethylenediamine, glucosamine, methylglucamine, purines, piperazines, piperidines, caffeine, procaine salts.

Pharmaceutically acceptable carriers can include one or several of the following compounds in a non limitating manner: fillers, such as sugar, starch, gelatine, gum, cellulose derivatives (methyl cellulose, hydroxy propylmethyl cellulose), polyvinylpyrrolidone, agar, alginic acid, talc, polyethylene glycol, pharmaceutically acceptable pigments and dyes, stabilizers, oils, liquid paraffin, ethanol, glycerids.

Preparation of Molecules

Another object of the instant invention is a process for the synthesis of the molecules of formula (I).

This process can be described by scheme 1 here-under:

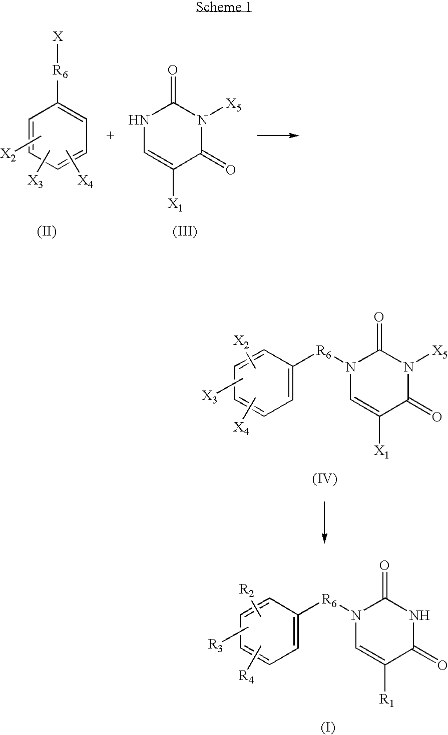

According to the process of the instant invention a haloaryl of formula (II) is reacted with a thymine or thymine derivative or uracyle or uracyle derivative of formula (III) to give the condensate (IV). In formula (II): X represents a halogen atom, preferentially Br; X₂, X₃, X₄ are selected among R₂, R₃ and R₄ respectively and their chemical precursors. In formula (III) and in formula (IV), X₁ is selected among R₁ and its chemical precursors, X₅ is selected among H and the benzyl group (Bzl).

By chemical precursors (X₁, X₂, X₃ and X₄) is meant a functional group which can be transformed in one or more steps into the desired functional group (R₁, R₂, R₃ and R₄).

As an example, if R₁ is H, or halogen, then X₁ is respectively H or said halogen, but if R₁ is —CH₂—COOH, then X₁ can be —CH₂—COOBz1, or —CH₂—OH, or —CH₂—Br, so that the alkylation of the thymine cycle can be effected in the absence of any side reaction.

In a second step and if necessary, X₁, X₂, X₃, X₄ and X₅ are transformed into R₁, R₂, R₃, R₄ and H respectively to give the molecule of formula (I).

According to a favourite variant of the invention, R₂═R₃═H and R₆═CH₂. Then a key intermediate molecule for the synthesis of the molecules of formula (I) is:

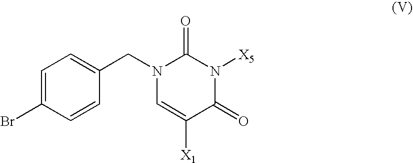

wherein X₁ and X₅ have the same definition as explained above.

And especially in the case when R₁ is CH₃, favourite intermediate molecules (V) for the preparation of other molecules of formula (I) are:

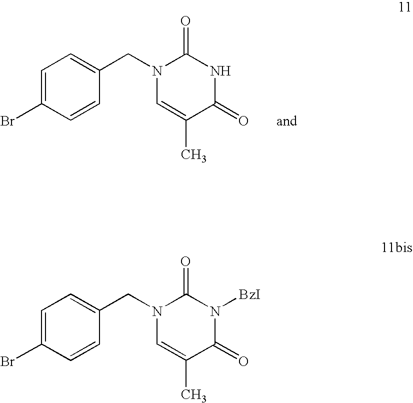

The use of a compound of formula (V), and especially compounds 11 and 11bis for the preparation of a molecule responding to formula (I) is another object of the invention.

The above-explained strategy is illustrated in the examples and in the schemes 2 and 3 here-under.

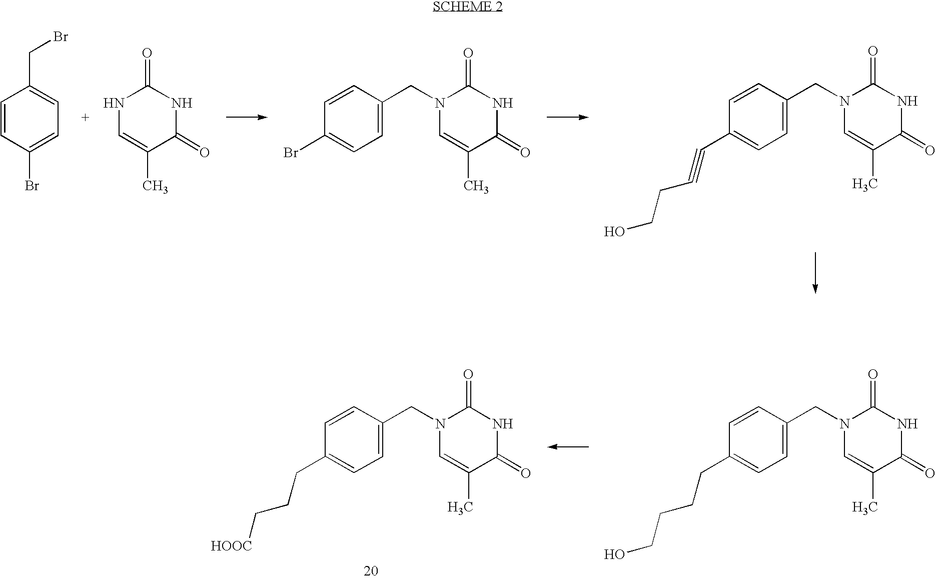

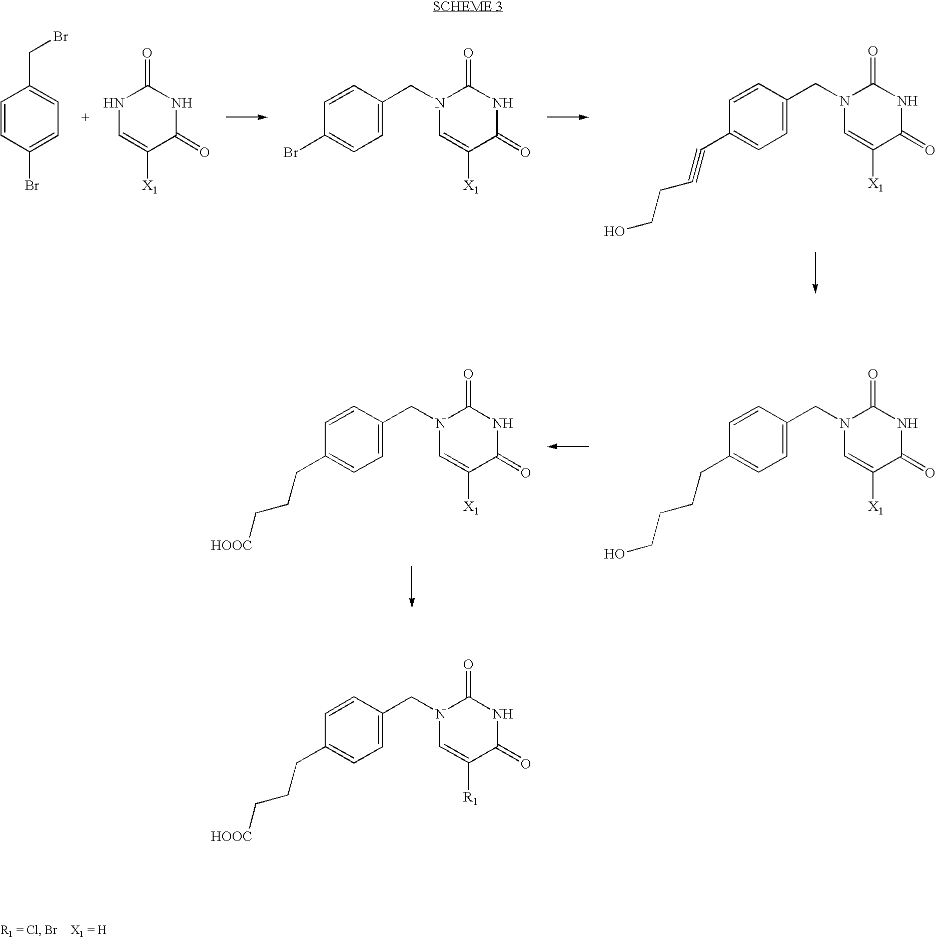

Favourite synthetic routes are based upon Heck or Sonogashira C—C palladium-catalysed coupling reactions between an aryl halide and a suitable alkene or alkyne (Scheme 4). Typically, the first step is the arylation of thymine or uracyle (N¹-benzoylated). Starting from this key halide intermediate (IV), various commercially available acids, esters or alcohols (depending on the selected chain length) were coupled using the Heck or Sonogashira reaction.

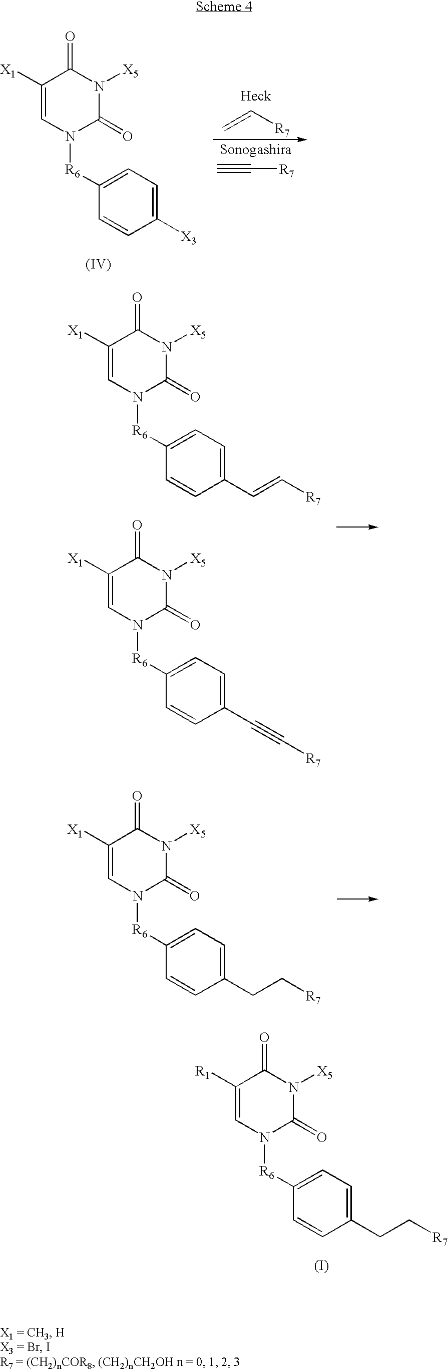

We considered for the preparation of N¹-benzyl thymine substituted by a C3 arm, the Heck palladium-catalyzed coupling reaction (Palladium-catalyzed reactions of organic halides with olefins, R. F. Heck et al, Accounts of Chemical Research, (1979), 12(4), 146-151) of an aryl bromide or iodide with an olefin as a precursor of the C3 chain (Scheme 5). Starting from the commercially available 4-iodo-benzyl bromide, N³-benzoylthymine was first alkylated to give 23 (88% yield) and then debenzoylated into iodide 24 (95% yield). A mixture of 24 and ethyl acrylate in anhydrous acetonitrile in the presence of Pd(OAc)₂ (2%), tri-o-toluoylphosphine (4%) and triethylamine was stirred at 90° C. for 18 h. The trans-olefin 17 was isolated in 97% yield. Compound 17 allowed the obtention of the target molecules 1 and 2, and also served as a precursor for the synthesis of three other related derivatives (14, 18, 19). Thus, hydrogenation on 10% Pd/C of the acrylic ethyl ester 17 afforded the propionic ethyl ester 19 (91% yield). Treatment of 19 in 1N NaOH afforded the propionic acid 2 (59% yield), while treatment of 19 in 35% aqueous ammonia gave the target compound 1 (90% yield). In the same manner, saponification of 17 afforded the propenacid derivative 18, while ammonolysis of 17 afforded the propenamide 14 in similar yields.

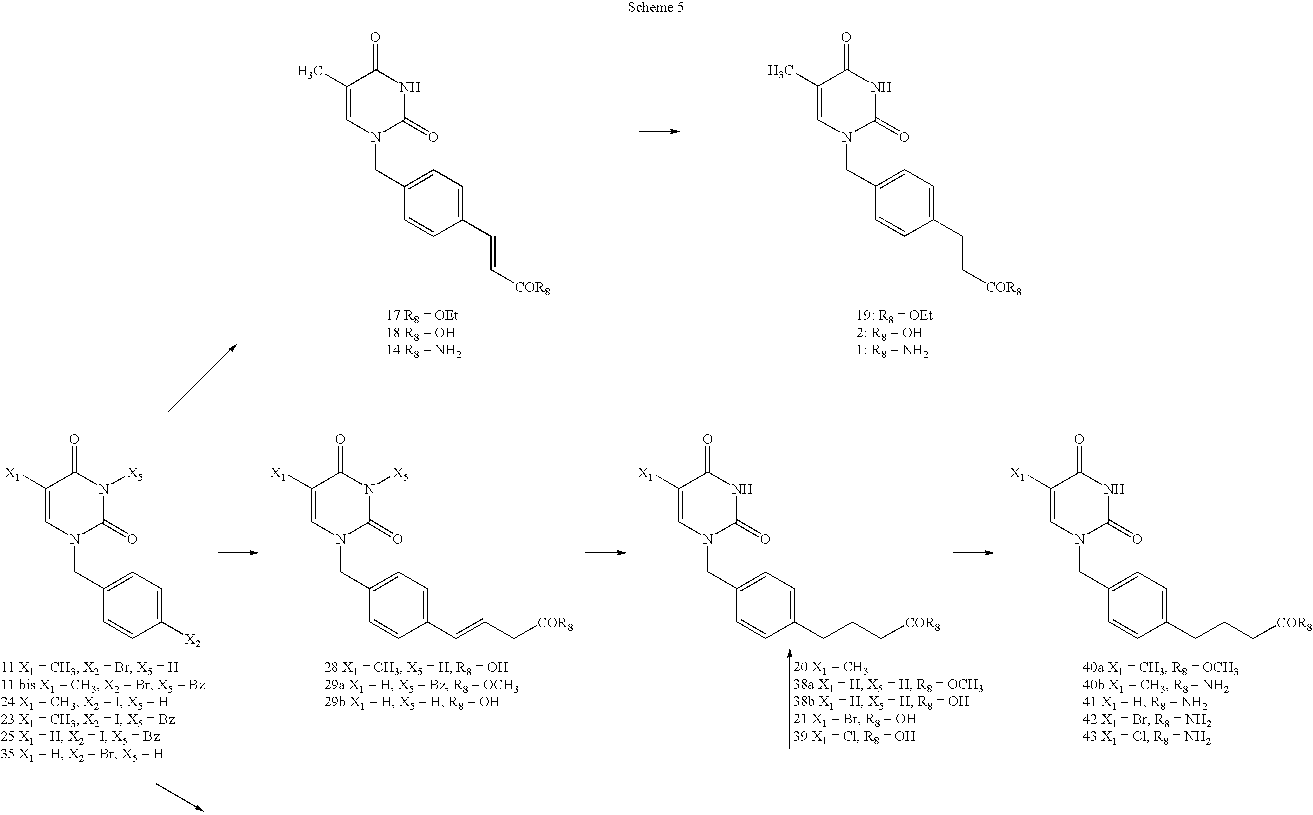

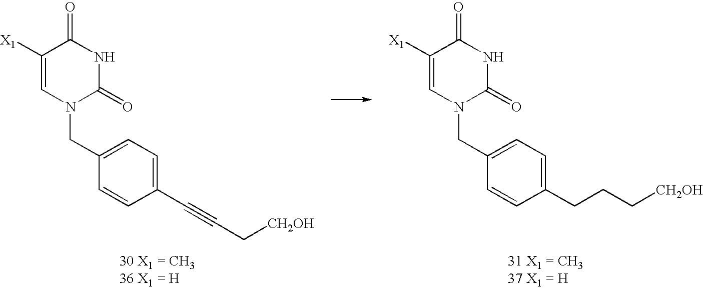

The synthesis of N¹-benzyl-thymine substituted in para by a C4 chain was depicted in Scheme 5. Two routes were investigated in accordance with the precursor chosen for introducing the carboxylic acid function. To obtain compound 20 according to the Heck-coupling reaction, a mixture of 24 and 3-butenoic acid in the presence of Pd(OAc)₂ (2%), tri-o-toluoylphosphine (4%) in acetonitrile and triethylamine was heated at 60° C. Olefin 28 was isolated in 27% yield while iodide 24 was recovered. Only one stereoisomer (Z-3, 4) was isolated as confirmed by NMR analysis. Raising the temperature to 90° C. did not improve the yield. When arylation was conducted starting from iodide 23, the coupling yield was slightly improved due to a facilitated purification of the corresponding resulting N¹-benzoylated derivative 28 (39%). The alkene 28 was then reduced with H₂ on 10% Pd—C to give C4 acid 20 (97% yield). Alternatively, compound 20 was obtained by the C—C coupling of 24 and a commercially available alkyne (Sonogashira reaction). Thus, but-3-yn-1-ol and iodide 24 in the presence of tetrakis(triphenylphosphine)palladium (6%), copper(I) iodide (2%) was refluxed at 90° C. in a mixture of CH₂Cl₂ and Et₃N under argon for 72 h. Compound 30 was isolated as the major arylation product in 40% yield. When the arylation was conducted at 60° C. for 72 h, 30 was isolated in 49% yield. Alkynol 30 was then reduced with H₂ on 10% Pd—C to give alcohol 31 (98% yield). Oxidation with PDC in the presence of t-butanol, followed by acid hydrolysis of the intermediate t-butyl ester gave the C4 acid 20 (46% yield in two steps). Reaction of acid 20 in refluxing methanol in the presence of Dowex H⁺ followed by treatment of the resulting methyl ester 40a with ammonia in methanol afforded amide 40b (58% yield in two steps).

For the synthesis of uracyl derivatives, the same sequences were followed starting from 4-halogenobenzyl-uracyle. Thus, Heck coupling reaction of iodide 25 (obtained by alkylation of N¹-benzoyluracyle with 4-iodobenzyl bromide) and methyl 3-butynoate afforded methyl ester 29a in 42% yield. Saponification of 29a gave acid 29b, while catalytic hydrogenation of 29a and saponification afforded acid 38a. Alternatively, Sonogashira coupling of bromide 11 and but-3-yn-1-ol afforded alkyne 36 in good yield (64%). Catalytic reduction into alcohol 37, oxidation into t-butyl ester followed by acid hydrolysis gave uracyl acid derivative 38a (49% yield in three steps). Bromation and chloration of 38a afforded the corresponding 5-Br and 5-Cl derivatives, 21 (70% yield) and 39 (49% yield), respectively. Conversion of acid 38b via methyl ester 38a into amide 41 was followed by bromation or chloration to yield 5-bromo or 5-chloro uracyl derivative 42 and 43, respectively.

N¹-benzyl-thymine having a C5 chain ended with a carboxylic acid was obtained in two steps by coupling an aryl halide and a suitable C5 alkyne (Scheme 6). Heck reaction between bromide 11 and 4-pentenoic acid in the presence of Pd(OAc)₂ and tri-o-toluoylphosphine gave 44 in 48% yield as a mixture of three stereoisomers: 4-E-pentenoic acid (44a), 3-E-pentenoic acid (44b) and 2-E-pentenoic acid (44c) in a ratio of 5/2/1 according to NMR analysis. Starting from N³-benzoylated thymine bromide 11bis, the coupling reaction was slightly improved (63% yield) while the purification of the products was enhanced. After debenzoylation, 44 was obtained in a similar overall yield. Reduction of the isomeric mixture of olefins 44 yielded the saturated C5 acid 45 (55% yield). Similarly, N¹-benzyl-uracyle substituted with a C5 chain was obtained by using Heck coupling of N³-benzoylated uracyl iodide 25 and 4-pentenoic acid (61%), followed by debenzoylation and catalytic hydrogenation of olefins 46 afforded acid 47, which was brominated and chlorinated to give 5-bromo and 5-chloro derivatives 48 and 49, respectively.

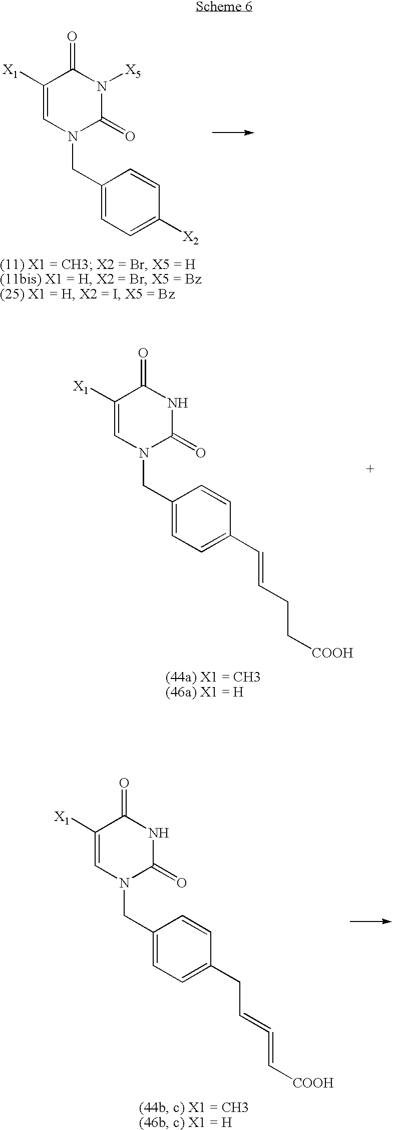

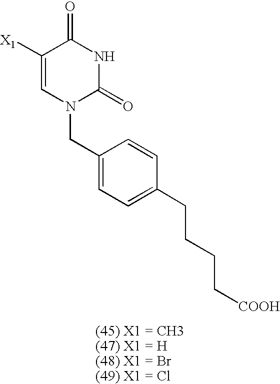

N-benzyl-thymine and 5-halogenated uracyle with a C6 chain were obtained by using the palladium-catalyzed Sonogashira reaction between an iodide and a suitable C6 alkyne ended with a carboxylic acid or alcohol. An example of each synthetic route is depicted in Scheme 7. In order to facilitate the purification of the arylation products, we started from the key intermediate 23 or 25 (when bases were N1-benzoylated). Thus, the palladium-catalyzed Sonogashira reaction between 23 and 5-hexynoic acid in the presence of tetrakis(triphenylphosphine)palladium (6%), copper(I) iodide (2%) gave 50 in 80% yield. Debenzoylation of 50 into 51, followed by hydrogenation resulted into saturated C6 acid 22 (50% yield in two steps). Conversion of acid 51 into amide 53 via the corresponding methyl ester 52, followed by reduction of 53 afforded C6 spacer amide 54 (42% yield in two steps). When 5-hexyn-1-ol was coupled with iodide 25, compound 55 was obtained in 70% yield. Debenzoylation followed by hydrogenation gave C6 alcohol 56 (83%). Chromic oxidation of 56 afforded C6 acid 57, which was brominated and chlorinated to give 5-bromo and 5-chloro-uracyl derivatives 58 and 59, respectively. Similarly, 5-halogenated uracyl derivatives ended with a C6 amide 61 and 62 were synthesized from amide 60 (obtained from acide 57).

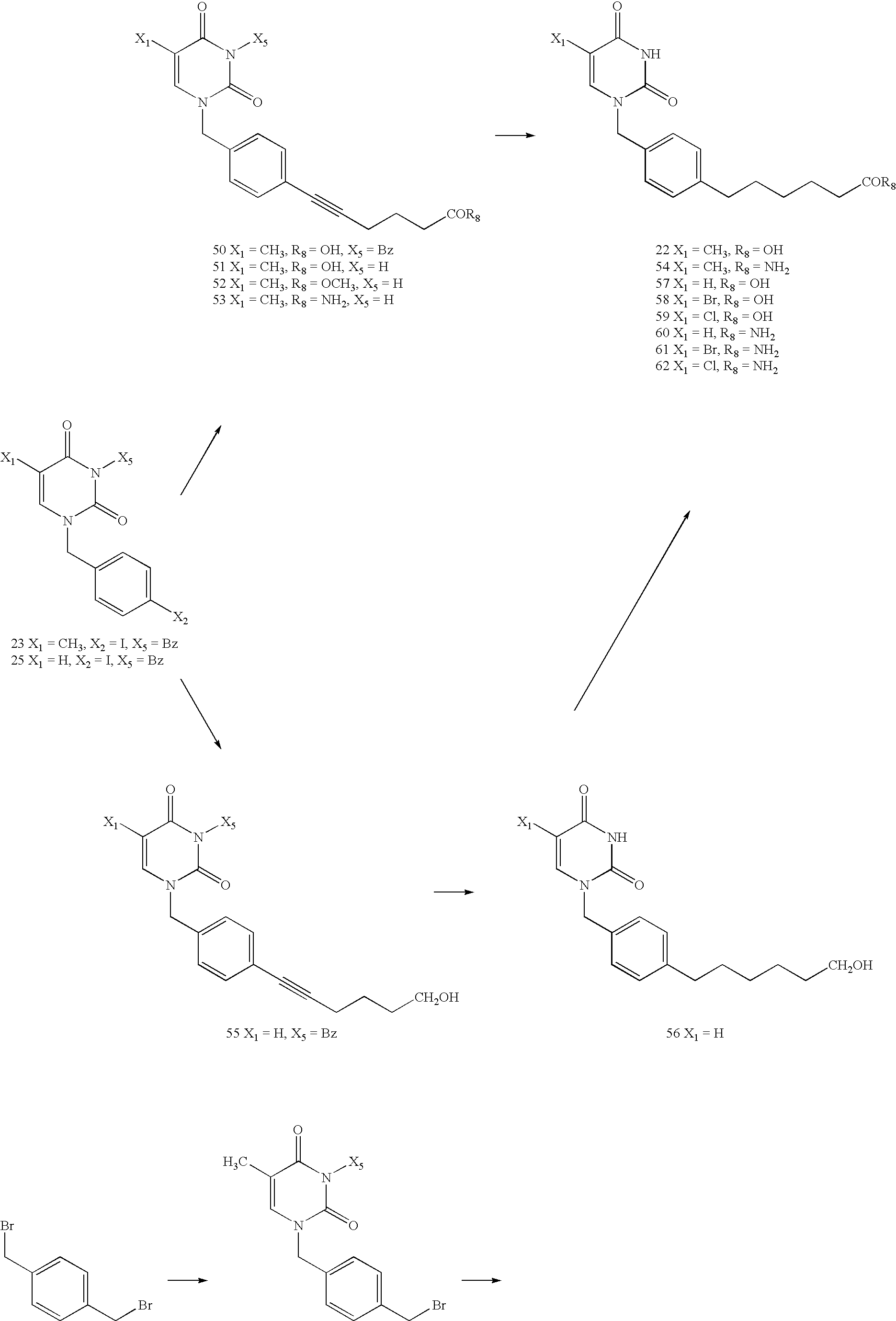

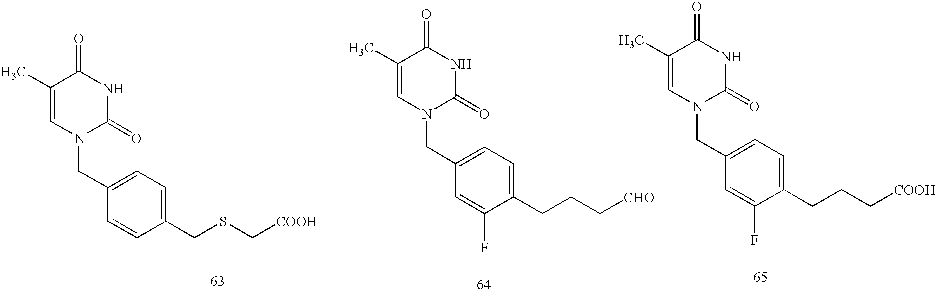

### EXPERIMENTAL SECTION

### A—Chemical Synthesis

General information Solvents were spectroscopic or HPLC grade. Reagents were purchased from Sigma-Aldrich or Fluka and used without purification. ¹H and ¹³C NMR spectra were recorded on a Bruker AC-400 spectrometer, operating at 400.13 MHz and 100.62 MHz, respectively. Chemical shifts are given in ppm (δ) relative to residual solvent peak for 1H and ¹³C, coupling constants (J) are reported in Hertz, and the normal abbreviations are used. Thin layer chromatography (TLC) was performed using Merck silica gel plates (Kieselgel 60 F₂₅₄/0.2 mm thickness) and spots were visualised by UV light, then revealed by sulfuric acid-anisaldehyde spray followed by heating. Column chromatography was performed with Merck silica gel 60 (230-400 mesh). Preparative HPLC were carried out on a Perkin Elmer system (200 Pump) with a C18 reverse phase column (Kromasil, 5 [symbol] 100 Å, 150×4.5 mm) using a flow rate of 5.5 ml/min and a linear gradient of CH₃CN (A) in 10 mM triethylammonium acetate buffer (B) at pH 7.5 over 20 min. Eluted products were visualized using a diode array detector. Purity of compounds was checked by anaytical HPLC on a C18 reverse phase column using a flow rate of 1 ml/min. ESI-TOF mass spectra were recorded by the mass spectroscopy laboratory (CNRS-ICSN, Gif-sur-Yvette).

General Procedure for N-alkylation (Scheme 2 and 3)

To a stirred solution of thymine (or related base) (100 mg) in DMF (4 ml) was added anhydrous K₂CO₃ (100 mg). A solution of aryl halide (1.1 eq.) in DMF (1 ml) was added over a period of 1 h, the stirring was maintained overnight at room temperature. The insoluble was removed by filtration and the filtrate was concentrated to dryness by co-evaporation with xylene. The crude residue was dissolved in CH₂Cl₂ and washed with water. The organic layer was dried, concentrated under vacuum and purified by column chromatography on silica gel (gradient of methanol in dichloromethane) to give N¹, N³-dialkylated and N¹-alkylated thymine in a 1:1.2-1.3 ratio.

Alternatively, alkylation of N³-benzoyl-thymine (obtained in two steps from thymine in 74% yield according to the method described in “The Benzoylation of Uracil and Thymine”, Cruickshank, K. A.; Jiricny, J.; Reese, C. B.; (1984) Tet. Letts., 25 (6), 681-684) afforded the N¹-alkylated N³-benzoyl-thymine as a unique product in 84% yield. Benzoyl deprotection under basic conditions (33% aqueous ammonia in methanol) results in the corresponding N⁴-alkylated thymine (nearly quant. yield).

### 5-Methyl-1-(4-bromo-benzyl)-1H-pyrimidine-2,4-dione (11)

Reaction of thymine (300 mg), K₂CO₃ (330 mg) with 4-bromo-benzyl bromide (1.1 eq) in DMF (15 ml) yielded after purification compound 11 (302 mg, 43% yield). ¹H NMR (CDCl₃) δ: 1.96 (d, 3H, CH₃, J=1.2 Hz), 4.85 (s, 2H, PhCH₂), 6.98 (d, 1H, H6, J=1.2 Hz), 7.19 (d, 2H, H arom., J=8 Hz), 7.51 (d, 2H, H arom.), 9.47 (bs, 1H, NH). ¹³C NMR (CDCl₃) δ: 12.76 (CH₃), 50.86 (PhCH₂), 111.98 (C5), 122.94 (C arom.), 130.07 and 132.63 (CH arom.), 134.93 (C arom.), 139.88 (C6), 151.59 (C2), 164.53 (C4). MS (ESI-TOF) m/z 295.0 and 297.0 (15%, M+H)⁺, 317.0 and 319.0 (20%, M+Na)⁺, 339.0 and 341.0 (100%, 85%, M+2Na)⁺.

### N³-Benzoyl-5-Methyl-1-(4-bromo-benzyl)-1H-pyrimidine-2,4-dione (11 bis)

Reaction of N³-benzoyl-thymine (230 mg, 1 mmol) with 4-bromo-benzyl bromide (140 mg) yielded after purification compound 11bis (336 mg, 84% yield). ¹H NMR (DMSO-d6) δ: 1.85 (d, 3H, CH₃, J=1.1 Hz), 4.90 (s, 2H, PhCH₂), 7.32 (d, 2H, H arom., J=8.4 Hz), 7.78 (m, 4H, H arom. and H arom. Bz), 7.78 (t, 1H, H arom. Bz), 7.93 (m, 2H, H arom. Bz), 7.95 (d, 1H, H6, J=1.1 Hz). ¹³C NMR (DMSO-d6) δ: 12.73 (CH₃), 51.14 (PhCH₂), 110.08 (C5), 121.93 (C arom.), 130.36, 130.71, 131.14 (CH arom.), 131.98 (C arom.), 132.49 and 136.32 (CH arom.), 136.64 (C arom.), 143.04 (C6), 150.38 (C2), 163.69 (C4), 170.46 (COBz). MS (ESI-TOF) m/z 421.0 and 423.0 (100% and 90%, M+Na).

### 1-[4-(4-Hydroxy-but-1-ynyl)-benzyl]-5-methyl-1H-pyrimidine-2,4-dione (30)

To a solution of compound 11 (1.94 g, 6.57 mmol) in dry CH₂Cl₂ (10 ml) were added under Ar freshly distilled Et₃N (27 ml), 3-butyn-1-ol (0.46 g, 6.57 mmol), tetrakis(triphenylphosphine)palladium (0.45 g, 0.39 mmol) and cuprous iodide (25 mg, 0.13 mmol). The reaction mixture was heated under reflux for 72 h. To the cooled mixture, CH₂Cl₂ (100 ml) was added and the resulting solution was washed with 5% aqueous citric acid (3×100 ml), then water. The organic layer was dried over Na₂SO₄, concentrated under vacuo. Purification by silica gel column chromatography (0-10% gradient of methanol in dichloromethane) afforded recovered starting material (0.52 g, 27%), then compound 30 as a white powder (0.44 g, 24%). ¹H NMR (DMSO-d6) δ: 1.75 (d, 3H, CH₃, J=1.2 Hz), 2.54 (t, 2H, CH₂), 3.58 (q, 2H, CH₂OH), 4.83 (s, 2H, PhCH₂), 4.92 (t, 1H, OH), 7.25 (d, 2H, H arom., J=8 Hz), 7.37 (d, 2H, H arom., J=8 Hz), 7.61 (d, 1H, H6, J=1.2 Hz), 11.33 (bs, 1H, NH). ¹³C NMR (DMSO-d6) δ: 12.77 (CH₃), 24.09 (CH₂), 50.72 (PhCH₂), 60.57 (CH₂), 81.54 (C alcyn), 89.68 (C alcyn), 109.98 (C5), 123.32 (C arom.), 128.40 (CH arom.), 132.38 (CH arom.), 137.63 (C arom.), 142.14 (C6), 151.84 (C2), 165.13 (C4). HRMS (ESI-TOF) m/z calculated for C₁₆H₁₇N₂O₃+Na 307.1059; found 307.1048.

### 1-[4-(4-Elydroxy-butyl)-benzyl]-5-methyl-1H-pyrimidine-2,4-dione (31)

To a solution of compound 30 (0.34 g, 1.19 mmol) in a mixture of CH₂Cl₂/methanol (1 ml/10 ml) was added Pd/C (100 mg). Hydrogen was applied overnight, then the mixture was passed through celite and the filtrate concentrated to dryness to give 31 after purification by silica gel column chromatography (0-10% gradient of methanol in dichloromethane) (0.24 g, 70% yield). ¹H NMR (DMSO-d6) δ: 1.42 (m, 2H, CH₂CH₂), 1.57 (m, 2H, CH), 1.75 (d, 3H, CH₃, J=1.2 Hz), 2.55 (t, 2H, CH₂Ph), 3.40 (m, 2H, CH₂OH), 4.36 (t, 1H, OH), 4.79 (s, 2H, PhCH₂), 7.11 (d, 2H, H arom., J=8.4 Hz), 7.20 (d, 2H, H arom., J=8.4 Hz), 7.61 (d, 1H, H6, J=1.2 Hz), 11.30 (bs, 1H, NH). ¹³C NMR (DMSO-d6) δ: 12.77 (CH₃), 28.27 (CH₂), 32.93 (CH₂), 35.47 (CH₂), 50.63 (PhCH₂), 61.36 (CH₂), 109.83 (C5), 128.29 (C arom.), 129.41 (CH arom.), 135.13 (CH arom.), 142.12 (C6), 142.67 (C arom.), 151.85 (C2), 165.09 (C4). HRMS (ESI-TOF) m/z calculated for C₁₆H₂₀N₂O₃+Na 311.1372; found 311.1374.

### 4-[4-(5-Methyl-2,4-dioxo-3,4-dihydro-2H-pyrimidin-1-yl-methyl)-phenyl]-butyric acid (20)

To a stirred solution of compound 31 (80 mg, 0.28 mmol) in dry CH₂Cl₂ (3 ml) were added t-butanol (0.54 ml, 5.6 mmol), acetic anhydride (0.26 ml, 2.8 mmol) and pyridinium dichromate (0.21 g, 0.56 mmol). After stirring for 1 h 30 at room temperature, starting material was consumed as judged by TLC. The crude mixture was loaded on a silica gel column chromatography conditioned in ethyl acetate, let on the silica gel for 15 min., then eluted with ethyl acetate. The fractions containing the expected ester were pooled, concentrated under vacuo and then treated with 2N NaOH (1 ml) in methanol (5 ml) overnight. The reaction mixture was acidified by addition of cationic resin (Dowex H⁺) until pH 3, filtered and lyophilized. Purification by reverse phase HPLC (5-30% linear gradient of acetonitrile in buffer, Rt=14 min.) gave compound 20 (20 mg, 25%). ¹H NMR (DMSO-d6) δ: 1.75 (d, 3H, CH₃, J=1.2 Hz), 1.78 (m, 2H, CH₂), 2.20 (t, 2H, CH₂), 2.57 (t, 2H, CH₂), 4.80 (s, 1H, H5), 7.19 (m, 4H, H arom.), 7.61 (d, 1H, H6, J=1.2 Hz), 11.30 (bs, 1H, NH). ¹³C NMR (DMSO-d₆) δ: 12.78 (CH₃), 27.12 (CH₂), 33.97 (CH₂), 34.90 (CH₂), 50.61 (PhCH₂), 109.83 (C5), 128.37 (CH arom.), 129.44 (CH arom.), 135.37 (C arom.), 141.92 (C arom.), 142.12 (C6), 151.86 (C2), 165.08 (C4), 175.09 (COOH). MS (ESI-TOF) m/z 325.1 (100%, M+Na)⁺. HRMS (ESI-TOF) m/z calculated for C₁₆H₁₈N₂O₄+Na 325.1178; found 325.0357.

### 1-(4-Bromo-benzyl)-1H-pyrimidine-2,4-dione (35)

A suspension of uracil (4.94 g, 44.0 mmol) and potassium carbonate (4.7 g, 33.9 mmol) in anhydrous DMF (60 ml) was stirred at room temperature for 1 h, then 4-bromobenzylmethyl bromide (8.47 g, 33.9 mmol) was added. After stirring overnight, the mixture was purified to give compound 35 (3.15 g, 33% yield). ¹H NMR (DMSO-d6) δ: 4.85 (s, 2H, PhCH₂), 5.60 (d, 1H, H5, J=7.8 Hz), 7.26 (d, 2H, H arom., J=8.4 Hz), 7.60 (dt, 2H, H arom., J=8.4 Hz, J=2 Hz), 7.76 (d, 1H, H6, J=7.8 Hz), 11.34 (bs, 1H, NH). ¹³C NMR (DMSO-d6) δ: 50.60 (PhCH₂), 102.32 (C5), 121.69 (C arom.), 130.58 CH arom.), 132.39 (CH arom.), 137.17 (CH arom.), 146.38 (C6), 151.84 (C2), 164.48 (C4). MS (ESI-TOF) m/z 281.0 and 283.0 (M+H)⁺, 303.0 and 305.0 (M+Na)⁺, 325.0 and 327.0 (M+2Na)⁺, 335.0 and 337.0.

### 1-(4-Hydroxy-but-1-ynyl-benzyl)-1H-pyrimidine-2,4-dione (36)

To a solution of compound 35 (1.46 g, 5.21 mmol) in dry CH₂Cl₂ (40 ml) were added under Ar freshly distilled Et₃N (45 ml), 3-butyn-1-ol (0.44 g, 6.57 mmol), tetrakis(triphenylphosphine)palladium (0.37 g, 0.31 mmol) and cuprous iodide (20 mg, 0.10 mmol). The reaction mixture was heated at 80° C. for 90 h and worked up as for compound 30 to give compound 36. ¹H NMR (DMSO-d6) δ: 1.19 (t, 21H, CH₃), 2.54 (t, 2H, CH₂), 3.10 (t, 14H, CH₂), 3.57 (q, 2H, CH₂), 4.87 (s, 2H, PhCH₂), 4.89 (t, 1H, OH), 5.60 (dd, 1H, H5, J=7.8 Hz, J=2.1 Hz), 7.25 (d, 2H, H arom., J=8.3 Hz), 7.38 (dd, 2H, H arom., J=6.5 Hz, J=2.7 Hz), 7.76 (d, 1H, H6), 9.10 (bs, 2.3H, NH), 11.33 (bs, 1H, NH). ¹³C NMR (DMSO-d6) δ: 24.12 (CH₂), 50.88 (PhCH₂), 60.56 (CH₂), 81.52 (C alcyn), 89.72 (C alcyn), 102.26 (C5), 123.36 (C arom.), 128.41 (CH arom.), 132.38 (CH arom.), 137.50 (C arom.), 146.38 (C6), 151.85 (C2), 164.50 (C4). MS (ESI-TOF) m/z 293.1 (100%, M+Na)⁺, 333.2 (5%, M+Na⁺ K)⁺.

### 1-[4-(4-1Hydroxy-butyl)-benzyl]-1H-pyrimidine-2,4-dione (37)

Compound 36 (0.55 g, 2.0 mmol) was hydrogenated as for compound 30 to give after purification compound 37 (0.25 g, 46%). ¹H NMR (DMSO-d6) δ: 1.41 (m, 2H, CH₂), 1.57 (m, 2H, CH₂), 2.55 (t, 2H, CH₂), 3.39 (m, 2H, CH₂), 4.35 (t, 1H, OH), 4.83 (s, 2H, PhCH₂), 5.58 (dd, 1H, H5, J=7.8 Hz, J=2.3 Hz), 7.19 (m, 4H, H arom.), 7.74 (d, 1H, H6, J=7.8 Hz), 11.30 (bs, 1H, NH). ¹³C NMR (DMSO-d6) δ: 28.26 (CH₂), 32.93 (CH₂), 35.46 (CH₂), 50.84 (PhCH₂), 61.34 (CH₂OH), 102.14 (C5), 128.40 (CH arom.), 129.42 (CH arom.), 134.95 (C arom.), 142.74 (C arom.), 146.45 (C6), 151.86 (C2), 164.50 (C4). MS (ESI-TOF) m/z 297.1 (100%, M+Na)⁺, 431.4 (40%).

4-[4-(2,4-dioxo-3,4-dihydro-2H-pyrimidin-1-ylmethyl)-phenyl]-butyric acid (38b) Compound 38b was obtained in two steps: chromic oxidation of compound 37 (135 mg, 0.5 mmol) as described for compound 32 gave the corresponding t-butyl ester (125 mg, 70%). Saponification of the ester (105 mg, 0.29 mmol) afforded compound 38b as a white powder (68 mg, 80%). ¹H NMR (DMSO-d6) δ: 1.77 (m, 2H, CH₂), 2.21 (t, 2H, CH₂), 2.57 (t, 2H, CH₂), 4.83 (s, 2H, PhCH₂), 5.58 (dd, 1H, H5, J=2.2 Hz and J=7.8 Hz), 7.20 (m, 4H, H arom.), 7.74 (d, 1H, H6, J=8.8 Hz), 11.30 (bs, 1H, NH), 12.05 (bs, 1H, COOH). ¹³C NMR (DMSO-d6) δ: 27.08 (CH₂), 33.91 (CH₂), 34.89 (CH₂), 50.83 (PhCH₂), 102.15 (C5), 128.40 (CH arom.), 129.46 (CH arom.), 135.19 (C arom.), 141.97 (C arom.), 146.44 (C6), 151.86 (C2), 164.50 (C4), 175.04 (COOH). HRMS (ESI-TOF) m/z calculated for C₁₅H₁₆N₂O₄+Na 311.1008; found 311.1022.

### 4-(5-Bromo-2,4-dioxo-3,4-dihydro-2H-pyrimidin-1-ylmethyl)-butyric acid (21)

To compound 38b (50 mg, 0.17 mmol) in pyridine (3 ml) was added a 1M solution of bromine in CCl₄ (0.23 ml). After stirring for 1 h30 reaction was complete, the solution was concentrated under vacuo. Purification by silica gel column chromatography (0-20% gradient of methanol in dichloromethane) afforded compound 21 as a pale yellow powder (45 mg, 70%). ¹H NMR (DMSO-d6) δ: 1.77 (m, 2H, CH₂), 2.21 (t, 2H, CH₂, J=7.3 Hz), 2.57 (t, 2H, CH₂), 4.84 (s, 2H, PhCH₂), 7.18 (d, 2H, H arom., J=8.1 Hz), 7.25 (d, 2H, H arom.), 8.35 (d, 1H, H6), 11.83 (bs, 1H, NH), 12.05 (bs, 1H, COOH). ¹³C NMR (DMSO-d6) δ: 27.08 (CH₂), 33.92 (CH₂), 34.90 (CH₂), 51.32 (PhCH₂), 95.92 (C5), 128.51 (CH arom.), 129.45 (CH arom.), 134.84 (C arom.), 142.09 (C arom.), 146.02 (C6), 151.22 (C2), 160.44 (C4), 175.04 (COOH). MS (ESI-TOF) m/z 389.0 (100%, M+Na)⁺, 391.0 (86%, M+Na)⁺. HRMS (ESI-TOF) m/z calculated for C₁₁H₁₅N₂O₄⁷⁹Br+Na 389.0113 and C₁₅H₁₅N₂O₄⁸¹Br+Na 391.0092; found 389.0140 (100%) and 391.0136 (87%).

### 5-Methyl-1-Benzyl-1H-pyrimidine-2,4-dione (3)

Reaction of thymine with benzyl bromide yielded after purification compound 3 (57 mg, % yield). ¹H NMR (CDCl₃) δ: 1.90 (d, 3H, CH₃, J=1.1 Hz), 4.92 (s, 2H, PhCH₂), 7.00 (d, 1H, H6, J=1.1 Hz), 7.31 (m, 2H, H arom.), 7.39 (m, 3H, H arom.), 9.14 (bs, 1H, NH). MS (ESI-TOF) m/z 217.1 (60%, M+H)⁺, 239.1 (100%, M+Na)⁺.

### 5-Bromo-1-benzyl-1H-pyrimidine-2,4-dione (4)

Reaction of 5-bromo-uracil with benzyl bromide yielded after purification compound 4. ¹H NMR (CDCl₃) δ: 4.91 (s, 2H, PhCH₂), 7.30-7.40 (m, 5H, H arom.), 8.37 (s, 1H, H6), 11.84 (bs, 1H, NH).

### 5-Fluoro-1-benzyl-1H-pyrimidine-2,4-dione (5)

Reaction of 5-fluoro-uracil with benzyl bromide yielded after purification compound 5. ¹H NMR (DMSO-d6) δ: 4.89 (s, 2H, PhCH₂), 7.34 (m, 5H, H arom.), 8.37 (s, 1H, H6), 11.84 (bs, 1H, NH). MS (ESI-TOF) m/z 279 (M+2Na).

### 5-Methyl-1-(4-fluoro-benzyl)-1H-pyrimidine-2,4-dione (6)

Reaction of thymine with 4-fluoro-benzyl chloride yielded after purification compound 6 (64 mg, 35% yield). ¹H NMR (CDCl₃) δ: 1.89 (d, 3H, CH₃, J=1.1 Hz), 4.87 (s, 2H, PhCH₂), 6.99 (d, 1H, H6, J=1.1 Hz), 7.06 (m, 2H, H arom.), 7.30 (m, 2H, H arom.), 9.75 (bs, 1H, NH). ¹³C NMR (CDCl₃) δ: 12.75 (CH₃), 50.76 (PhCH₂), 111.87 (C5), 116.18 and 116.52 (CH arom.), 130.25 and 130.33 (CH arom.), 131.78 and 131.81 (C arom.), 139.95 (C6), 151.73 (C2), 161.84 and 161.30 (C arom.), 164.74 (C4). MS (ESI-TOF) m/z 235.1 (90%, M+H)⁺, 257.1 (40%, M+Na)⁺, 279.1 (50%, M+2Na)⁺.

### 5-Methyl-1-(3-fluoro-benzyl)-1H-pyrimidine-2,4-dione (7)

Reaction of thymine with 3-fluoro-benzyl chloride yielded after purification compound 7 (48 mg, 26% yield). ¹H NMR (CDCl₃) δ: 1.90 (d, 3H, CH₃, J=1.1 Hz), 4.90 (s, 2H, PhCH₂), 7.05 (m, 3H, H6 and H arom.), 7.08 (d, 1H, H arom.), 7.35 (m, 1H, H arom.), 9.75 (bs, 1H, NH). ¹³C NMR (CDCl₃) δ: 12.75 (CH₃), 50.86 (PhCH₂), 111.98 (C5), 115.18 and 115.40 (CH arom.), 115.69 and 115.90 (CH arom.), 123.83 and 123.86 (CH arom.), 131.06 and 131.14 (CH arom.), 138.36 and 138.44 (C arom.), 140.00 (C6), 151.68 (C2), 162.19 (C4), 164.66 and 164.72 (C arom.). MS (ESI-TOF) m/z 235.1 (100%, M+H)⁺, 257.1 (5%, M+Na)⁺, 279.1 (70%, M+2Na)⁺.

### 5-Methyl-1-(3,4-difluoro-benzyl)-1H-pyrimidine-2,4-dione (8)

Reaction of thymine with 3,4-difluoro-benzyl bromide yielded after purification compound 8 (60 mg, 30% yield). ¹H NMR (CDCl₃) δ: 1.89 (d, 3H, CH₃, J=1.1 Hz), 4.90 (s, 2H, PhCH₂), 5.30 (d, 1H, ═CH, J=11 Hz), 5.77 (d, 1H, ═CH, J=18 Hz), 6.72 (dd, 1H, ═CH, J=11 Hz, J=18 Hz), 6.98 (d, 1H, H6, J=1.1 Hz), 7.27 (m, 2H, H arom., J=8 Hz), 7.42 (m, 2H, H arom.), 9.26 (bs, 1H, NH). ¹³C NMR (CDCl₃) δ: 12.74 (CH₃), 50.61 (PhCH₂), 112.15 (C5), 117.47 and 117.64 (CH arom.), 118.25 and 118.43 (CH arom.), 124.46, 124.49, 124.52 and 124.56 (CH arom.), 132.85 and 132.90 (C arom.), 139.76 (C6), 149.46, 149.58, 149.63 and 149.75 (C arom.), 151.49 (C2), 151.93, 152.06, 152.11 and 152.24 (C arom.), 164.48 (C4). MS (ESI-TOF) m/z 253.1 (100%, M+H)⁺.

### 5-Methyl-1-(4-chloro-benzyl)-1H-pyrimidine-2,4-dione (9)

Reaction of thymine with 4-chloro-benzyl chloride (1.1 eq) yielded after purification compound 8 (71 mg, 36% yield). ¹H NMR (CDCl₃) δ: 1.89 (d, 3H, CH₃, J=1 Hz), 4.86 (s, 2H, PbCH₂), 6.99 (d, 1H, H6), 7.25 (d, 2H, H arom.), 7.34 (d, 2H, H arom.), 9.88 (bs, 1H, NH). ¹³C NMR (CDCl₃) δ: 12.75 (CH₃), 50.80 (PhCH₂), 111.96 (C5), 126.65 and 129.77 (CH arom.), 134.44 and 134.82 (C arom.), 139.92 (C6), 151.68 (C2), 164.66 (C4). MS (ESI-TOF) m/z 251.1 (20%, M+H)⁺, 295.0 (100%, M+2Na)⁺.

### 5-Methyl-1-(3-chloro-benzyl)-1H-pyrimidine-2,4-dione (10)

Reaction of thymine with 3-chloro-benzyl chloride (1.1 eq) yielded after purification compound (52 mg, 26% yield). ¹H NMR (CDCl₃) δ: 1.92 (d, 3H, CH₃, J=1.1 Hz), 4.88 (s, 2H, PhCH₂), 6.99 (d, 1H, H6, J=1.1 Hz), 7.20 (m, 1H, H arom.), 7.28 (m, 1H, H arom.), 7.32 (m, 2H, H arom.), 9.45 (bs, 1H, NH). ¹³C NMR (CDCl₃) δ: 12.77 (CH₃), 50.85 (PhCH₂), 112.03 (C5), 126.44, 128.37, 129.06 and 130.79 (CH arom.), 135.36 (C arom.), 137.92 (C6), 139.92 (C arom.), 151.55 (C2), 164.52 (C4). MS (ESI-TOF) m/z 251.1 (100%, M+H)⁺, 273.0 (25%, M+Na)⁺, 295.0 (35%, M+2Na)⁺.

### 5-Methyl-1-(4-bromo-benzyl/-1H-pyrimidine-2,4-dione (11)

Reaction of thymine (300 mg), K₂CO₃ (330 mg) with 4-bromobenzyl bromide (1.1 eq) in DMF (15 ml) yielded after purification compound (302 mg, 43% yield).

¹H NMR (DMSO-d6) δ: 1.75 (d, 3H, CH₃, J=1.2 Hz), 4.81 (s, 2H, PhCH₂), 7.26 (d, 2H, H arom., J=8.3 Hz), 7.56 (d, 2H, H arom., J=8.3 Hz), 7.64 (d, 1H, H6, J=1.2 Hz), 11.34 (bs, 1H, NH). ¹³C NMR (DMSO-d6) δ: 12.79 (CH₃), 50.37 (PhCH₂), 109.99 (C5), 121.63 (C arom.), 130.57 and 132.38 (CH arom.), 137.34 (C arom.), 142.03 (C6), 151.83 (C2), 165.07 (C4). MS (ESI-TOF) m/z 295.0 and 297.0 (15%, M+H)⁺, 317.0 and 319.0 (20%, M+Na)⁺, 339.0 and 341.0 (100%, 85%, M+2Na)⁺.

3-[4-(5-Methyl-2,4-dioxo-3,4-dihydro-2H-pyrimidin-1-ylmethyl)-phenyl]-acrylic acid ethyl ester (17). ¹H NMR (DMSO-d6) δ: 1.26 (t, 3H, CH₃), 1.76 (d, 3H, CH₃, J=1.1 Hz), 4.19 (q, 2H, OCH₂), 4.86 (s, 2H, PhCH₂), 6.62 (d, 1H, ═CH, J=16 Hz), 7.32 (d, 2H, H arom., J=8.1 Hz), 7.63 (d, 1H, ═CH), 7.64 (d, 1H, H6, J=1.1 Hz), 7.71 (d, 2H, H arom.), 11.33 (bs, 1H, NH). ¹³C NMR (CDCl₃) δ: 12.79 (CH₃), 15.04 (CH₂CH₃), 50.72 (PhCH₂), 60.97 (OCH₂), 109.98 (C5), 119.13 (═CH), 128.75 and 129.49 (CH arom.), 134.26 (C arom.), 140.27 (C arom.), 142.27 (C6), 144.70 (═CH), 151.86 (C2), 165.09 (C4), 167.01 (CO). MS (ESI-TOF) m/z 315.1 (5%, M+H)⁺, 337.1 (100%, M+Na)⁺, 359.1 (5%, M+2Na)⁺.

3-[4-(2,4-Dioxo-3,4-dihydro-2H-pyrimidin-1-yl-methyl)-phenyl]-acrylic acid (18). Compound 17 (50 mg, 0.16 mmol) was treated with NaOH (1.3 eq.) for 1 h. The mixture was passed through a Dowex H⁺ eluted with water. Compound 18 was isolated as a white powder (27 mg, 59% yield). ¹H NMR (DMSO-d6) δ: 1.76 (d, 3H, CH₃, J=1.2 Hz), 4.86 (s, 2H, PhCH₂), 6.52 (d, 1H, ═CH, J=16 Hz), 7.32 (d, 2H arom., J=8.2 Hz), 7.57 (d, 1H, ═CH), 7.64 (s, 1H, H6), 7.68 (d, 2H, H arom.), 11.34 (bs, 1H, NH), 12.40 (bs, 1H, COOH). ¹³C NMR (DMSO-d6) δ: 13.96 (CH₃), 51.87 (PhCH₂), 111.14 (C5), 121.37 (═CH), 129.93 (CH arom.), 130.52 (CH arom.), 135.65 (C arom.), 141.19 (C arom.), 143.28 (C6), 145.42 (═CH), 153.04 (C2), 166.26 (C4), 169.54 (COOH). HRMS (ESI-TOF) 7 m/z calculated for C₁₅H₁₃N₂O₄ 285.0875; found 285.0891.

3-[4-(5-Methyl-2,4-dioxo-3,4-dihydro-2H-pyrimidin-1-ylmethyl)-phenyl]-acrylamide (14). ¹H NMR (DMSO-d6) δ: 1.76 (d, 3H, CH₃, J=1.2 Hz), 4.82 (s, 2H, PhCH₂), 6.59 (d, 1H, ═CH, J=16 Hz), 7.09 (bs, 1H, CONH₂), 7.32 (d, 2H, H arom., J=8.2 Hz), 7.40 (d, 1H, ═CH), 7.55 (bd, 3H, H arom. and CONH₂), 7.64 (s, 1H, H6), 11.32 (bs, 1H, NH). ¹³C NMR (DMSO-d6) δ: 12.79 (CH₃), 50.69 (PhCH₂), 109.87 (C5), 123.30 (═CH), 127.50 (CH arom.), 128.23, 128.68 and 128.80 (CH arom.), 135.12 (C arom.), 139.18 (C arom.), 139.48 (═CH), 142.12 (C6), 151.86 (C2), 165.09 (C4), 167.45 (CONH₂). MS (ESI-TOF) m/z 285.2 (75%) 286.2 (100%, M+H)⁺, 307.1 308.1 (40%, M+Na)⁺.

3-[4-(5-Methyl-2,4-dioxo-3,4-dihydro-2H-pyrimidin-1-ylmethyl)-phenyl]-propionic acid ethyl ester (19). To compound 17 (90 mg, 0.28 mmol) in methanol (10 ml) was added Pd black (9 mg). Hydrogen was applied overnight, then the mixture was passed through celite and the filtrate concentrated to dryness to give 19 (83 mg, 91% yield). ¹H NMR (DMSO-d6) δ: 1.14 (t, 3H, CH₃), 1.74 (d, 3H, CH₃, J=1 Hz), 2.59 (t, 2H, CH₂, J=7.5 Hz), 2.82 (t, 2H, CH₂), 4.03 (q, 2H, OCH₂, J=7 Hz), 4.79 (s, 2H, PhCH₂), 7.20 (bs, 4H, H arom.), 7.60 (d, 1H, H6, J=1.0 Hz), 11.29 (bs, 1H, NH). ¹³C NMR (DMSO-d6) δ: 12.79 (CH₃), 14.92 (OCH₂CH₃), 30.77 (CH₂), 35.82 (CH₂), 50.59 (PhCH₂), 60.67 (OCH₂), 109.82 (C5), 128.38 (CH arom.), 129.38 (CH arom.), 135.67 (C arom.), 140.83 (C arom.), 142.06 (C6), 151.92 (C2), 165.17 (C4), 172.97 (COOEt). MS (ESI-TOF) m/z 339.1 (100%, M+Na)⁺.

3-[4-(5-Methyl-2,4-dioxo-3,4-dihydro-2H-pyrimidin-1-ylmethyl)-phenyl]-propionic acid (2). Compound 19 (90 mg, 0.28 mmol) was treated with 0.5 N NaOH (1.3 eq.) for 1 h. The mixture was passed through a Dowex H⁺ eluted with water. Compound 2 was isolated as a white powder (27 mg, 59% yield). ¹H NMR (DMSO-d6) δ: 1.75 (d, 3H, CH₃, J=1.0 Hz), 2.47 (t, 2H, CH₂), 2.79 (t, 2H, CH₂, J=7.7 Hz), 4.70 (s, 2H, PhCH₂), 7.15 (d, 4H arom.), 7.65 (d, 1H, H6), 11.30 (bs, 1H, NH). ¹³C NMR (DMSO-d6) δ: 12.77 (CH₃), 31.09 (CH₂), 36.43 (CH₂), 50.58 (PhCH₂), 109.84 (C5), 128.34 and 129.37 (CH arom.), 135.45 and 141.48 (C arom.), 142.10 (C6), 151.85 (C2), 165.07 (C4). HRMS (ESI-TOF) m/z calculated for C₁₅H₁₆N₂O₄+Na 311.1008; found 311.1029.

3-[4-(5-Methyl-2,4-dioxo-3,4-dihydro-2H-pyrimidin-1-ylmethyl)-phenyl]-propionamide (1). Compound 19 (70 mg, 0.22 mmol) was treated with 33% aqueous ammonia (25 ml) at room temperature overnight. Purification by silica gel column chromatography (0-8% gradient of methanol in dichloromethane) afforded compound 1 as a white powder (57 mg, 90%). ¹H NMR (DMSO-d6) δ: 1.75 (d, 3H, CH₃, J=1 Hz), 2.33 (m, 2H, CH₂), 2.79 (m, 2H, CH₂), 4.79 (s, 1H, H5), 6.74 (bs, 1H, CONH₂), 7.20 (m, 4H, H arom.), 7.28 (bs, 1H, CONH₂), 7.60 (d, 1H, H6, J=1 Hz), 11.29 (bs, 1H, NH). ¹³C NMR (DMSO-d6) X: 12.77 (CH₃), 31.25 (CH₂), 37.41 (CH₂), 50.59 (PhCH₂), 109.83 (C5), 128.33 (CH arom.), 129.33 (CH arom.), 135.37 (C arom.), 141.82 (C arom.), 142.09 (C6), 151.85 (C2), 165.07 (C4), 174.18 (CONH₂). MS (ESI-TOF) m/z 310.1 (100%, M+Na)⁺. HRMS (MALDI-TOF) m/z calculated for C₁₅H₁₇N₃O₃+Na 310.1168; found 310.1174.

### 4-[5-Bromo-(2,4-dioxo-3,4-dihydro-2H-pyrimidin-1-ylmethyl)-phenyl]butyric acid (21)

To compound 38b (50 mg, 0.17 mmol) in pyridine (3 ml) was added a 1M solution of bromine in CCl₄ (0.23 ml). After stirring for 1 h30 reaction was complete, the solution was concentrated under vacuo. Purification by silica gel column chromatography (0-20% gradient of methanol in dichloromethane) afforded compound 21 as a pale yellow powder (45 mg, 70%). ¹H NMR (DMSO-d6) δ: 1.77 (m, 2H, CH₂), 2.21 (t, 2H, CH₂, J=7.3 Hz), 2.57 (t, 2H, CH₂), 4.84 (s, 2H, PhCH₂), 7.18 (d, 2H, H arom., J=8.1 Hz), 7.25 (d, 2H, H arom.), 8.35 (d, 1H, H6), 11.83 (bs, 1H, NH), 12.05 (bs, 1H, COOH). ¹³C NMR (DMSO-d6) δ: 27.08 (CH₂), 33.92 (CH₂), 34.90 (CH₂), 51.32 (PhCH₂), 95.92 (C5), 128.51 (CH arom.), 129.45 (CH arom.), 134.84 (C arom.), 142.09 (C arom.), 146.02 (C6), 151.22 (C2), 160.44 (C4), 175.04 (COOH). MS (ESI-TOF) m/z 389.0 (100%, M+Na)⁺, 391.0 (86%, M+Na)⁺. HRMS (ESI-TOF) m/z calculated for C₁₅H₁₅N₂O₄⁷⁹Br+Na 389.0113 and C₁₅H₁₅N₂O₄⁸¹Br+Na 391.0092; found 389.0140 (100%) and 391.0136 (87%).

5-[5-Chloro-4-(2,4-dioxo-3,4-dihydro-2H-pyrimidin-1-yl-methyl)-phenyl]-butyric acid (39) ¹H NMR (DMSO-d6) δ: 1.77 (m, 2H, CH₂), 2.21 (t, 2H, CH₂, J=7.4 Hz), 2.57 (t, 2H, CH₂, J=7.7 Hz), 4.84 (s, 2H, PhCH₂), 7.15 (d, 2H, H arom., J=8.1 Hz), 7.25 (d, 2H, H arom., J=8.1 Hz), 8.29 (s, 1H, H6), 11.89 (bs, 11H, NH). ¹³C NMR (DMSO-d6) δ: 27.09 (CH₂), 33.92 (CH₂), 34.90 (CH₂), 51.37 (PhCH₂), 107.42 (C5), 128.49, 128.51 and 129.46 (CH arom.), 134.78 (C arom.), 142.10 (C arom.), 143.67 (C6), 150.99 (C2), 160.28 (C4), 175.05 (COOH). HRMS (ESI-TOF) m/z calculated for C₁₅H₁₅N₂O₄³⁵Cl+Na 321.0642; found 321.0638 (100%).

4-(5-Bromo-2,4-dioxo-3,4-dihydro-2H-pyrimidin-1-ylmethyl)-butyric acid amide (42) ¹H NMR (DMSO-d6) δ: 1.76 (m, 2H, CH₂), 2.05 (t, 2H, CH₂, J=7.5 Hz), 2.54 (t, 2H, CH₂, J=7.8 Hz), 4.84 (s, 2H, PhCH₂), 6.71 (bs, 1H, CONH₂), 7.18 (d, 2H, H arom., J=8.1 Hz), 7.24 (bs, 1H, CONH₂), 7.25 (d, 2H, H arom.), 8.36 (s, 1H, H6), 11.83 (bs, 1H, NH). ¹³C NMR (DMSO-d6) δ: 27.63 (CH₂), 35.18 (CH₂), 35.36 (CH₂), 51.32 (PhCH₂), 95.93 (C5), 128.49 (CH arom.), 129.46 (CH arom.), 134.76 (C arom.), 142.37 (C arom.), 146.01 (C6), 151.24 (C2), 160.47 (C4), 174.79 (CONH₂). HRMS (ESI-TOF) m/z calculated for C₁₅H₁₆N₃O₃⁷⁹Br+Na 389.0113 and C₁₅H₁₆N₃O₃⁸¹Br+Na 390.0252; found 388.0299 (100%) and 390.0282 (83%).

4-(5-Chloro-2,4-dioxo-3,4-dihydro-2H-pyrimidin-1-ylmethyl)-butyric acid amide (43) ¹H NMR (DMSO-d6) δ: 1.76 (m, 2H, CH₂), 2.05 (t, 2H, CH₂, J=7.4 Hz), 2.54 (t, 2H, CH₂, J=7.7 Hz), 4.84 (s, 2H, PhCH₂), 6.71 (bs, 1H, CONH₂), 7.18 (d, 2H, H arom., J=8.1 Hz), 7.24 (bs, 1H, CONH₂), 7.25 (d, 2H, H arom.), 8.29 (s, 1H, H6), 11.86 (bs, 1H, NH). ¹³C NMR (DMSO-d6) δ: 27.63 (CH₂), 35.18 (CH₂), 35.36 (CH₂), 51.37 (PhCH₂), 107.41 (C5), 128.49 (CH arom.), 129.46 (CH arom.), 134.69 (C arom.), 142.37 (C arom.), 143.67 (C6), 151.00 (C2), 160.28 (C4), 174.79 (CONH₂). HRMS (ESI-TOF) m/z calculated for C₁₅H₁₅N₂O₄³⁵Cl+Na 344.0778 and C₁₅H₁₅N₂O₄³⁷Cl⁺ Na 346.0748; found 344.0798 (100%) and 346.0798 (40%).

### 5-[4-(5-Methyl-2,4-dioxo-3,4-dihydro-2H-pyrimidin-1-yl-methyl)-phenyl]-penten-2-oic acid (28)

HRMS (ESI-TOF) m/z calculated for C₁₆H₁₆N₂O₄+Na 323.1008; found 232.1044.

5-[4-(5-Methyl-2,4-dioxo-3,4-dihydro-2H-pyrimidin-1-yl-methyl)-phenyl]-pentanoic acid (45) ¹H NMR (CDCl₃) δ: 1.67 (m, 4H, 2×CH₂), 1.90 (d, 3H, CH₃, J=1.1 Hz), 2.35 (t, 2H, CH₂, J=7.1 Hz), 2.65 (t, 2H, CH₂, J=7.1 Hz), 4.87 (s, 2H, PhCH₂), 6.99 (d, 1H, H6, J=1.2 Hz), 7.21 (m, 4H, H arom.), 8.74 (bs, 1H, NH). ¹³C NMR (CDCl₃) δ: 12.77 (CH₃), 24.90 (CH₂), 31.11 (CH₂), 34.26 (CH₂), 35.60 (CH₂), 51.12 (PhCH₂), 111.52 (C5), 128.47, 128.55 and 129.50 (CH arom.), 133.26 (C arom.), 140.15 (C arom.), 142.95 (C6), 151.42 (C2), 164.31 (C4), 174.39 (COOH). HRMS (ESI-TOF) m/z calculated for C₁₈H₂₂N₂O₄+Na 353.1477; found 353.1463.

### 5-[4-(2,4-dioxo-3,4-dihydro-2H-pyrimidin-1-yl-methyl)-phenyl]-pentanoic acid (47)

¹H NMR (DMSO-d6) δ: 1.52 (m, 4H, 2×CH₂), 2.22 (t, 2H, CH₂, J=7.1 Hz), 2.56 (t, 2H, CH₂, J=7.2 Hz), 4.83 (s, 2H, PhCH₂), 5.59 (d, 1H, H5, J=7.8 Hz), 7.20 (m, 4H, H arom.), 7.74 (d, 1H, H6, J=7.8 Hz), 11.31 (bs, 1H, NH). ¹³C NMR (DMSO-d6) δ: 24.98 (CH₂), 31.32 (CH₂), 34.37 (CH₂), 35.30 (CH₂), 50.83 (PhCH₂), 102.15 (C5), 128.34, 129.18 and 129.41 (CH arom.), 135.02 (C arom.), 142.42 (C arom.), 146.44 (C6), 151.87 (C2), 164.50 (C4), 175.28 (COOH).

5-[4-(5-Bromo-2,4-dioxo-3,4-dihydro-2H-pyrimidin-1-yl-methyl)-phenyl]-pentanoic acid (48) ¹H NMR (DMSO-d6) δ: 1.52 (m, 4H, 2×CH₂), 2.20 (t, 2H, CH₂, J=7.1 Hz), 2.56 (t, 2H, CH₂, J=7.2 Hz), 4.84 (s, 2H, PhCH₂), 7.19 (d, 2H, H arom.), 7.23 (d, 2H, H arom.), 8.35 (s, 1H, H6), 11.62 (bs, 1H, NH). ¹³C NMR (DMSO-d6) δ: 25.07 (CH₂), 31.25 (CH₂), 34.58 (CH₂), 35.33 (CH₂), 51.31 (PhCH₂), 95.93 (C5), 128.45 and 129.51 (CH arom.), 134.68 (C arom.), 142.58 (C arom.), 146.00 (C6), 151.25 (C2), 160.48 (C4), 175.43 (COOH). HRMS (ESI-TOF) m/z calculated for C₁₆H₁₇N₂O₄⁷⁹Br+Na 403.0269; found 403.0302.

### 6-[4-(5-Methyl-2,4-dioxo-3,4-dihydro-2H-pyrimidin-1-yl-methyl)-phenyl]-hexyn-5-oic acid (51)

HRMS (ESI-TOF) m/z calculated for C₁₈H₁₈N₂O₄+Na 349.1164; found 349.1160.

### 6-[4-(5-Methyl-2,4-dioxo-3,4-dihydro-2H-pyrimidin-1-yl-methyl)-phenyl]-hexyn-5-oic acid amide (53)

HRMS (ESI-TOF) m/z calculated for C₁₈H₁₉N₃O₃+Na 348.1324; found 348.1292.

### 6-[4-(5-Methyl-2,4-dioxo-3,4-dihydro-2H-pyrimidin-1-yl-methyl)-phenyl]-hexanoic acid (22)

¹³C NMR (DMSO-d6) δ: 12.78 (CH₃), 25.19 (CH₂), 29.08 (CH₂), 31.55 (CH₂), 34.50 (CH₂), 35.51 (CH₂), 50.62 (PhCH₂), 109.81 (C5), 128.31 (CH arom.), 129.39 (CH arom.), 135.15 (C arom.), 142.13 (C6), 142.56 (C arom.), 151.84 (C2), 165.07 (C4), 175.33 (COOH). HRMS (ESI-TOF) m/z calculated for calculated for C₁₈H₂₂N₃O₄+Na 353.1477; found 353.1455.

### 6-[4-(5-Methyl-2,4-dioxo-3,4-dihydro-2H-pyrimidin-1-yl-methyl)-phenyl]-hexanoic acid amide (54)

¹³C NMR (DMSO-d6) δ: 12.78 (CH₃), 25.77 (CH₂), 29.19 (CH₂), 31.60 (CH₂), 35.54 (CH₂), 35.88 (CH₂), 50.63 (PhCH₂), 109.81 (C5), 128.30 (CH arom.), 129.39 (CH arom.), 135.14 (C arom.), 142.15 (C6), 142.62 (C arom.), 151.85 (C2), 165.08 (C4), 175.11 (CONH₂). HRMS (ESI-TOF) m/z calculated for calculated for C₁₈H₂₃N₃O₃+Na 352.1637; found 352.1601.

### 6-[4-(5-Bromo-2,4-dioxo-3,4-dihydro-2H-pyrimidin-1-yl-methyl)-phenyl]-hexanoic acid (58)

¹H NMR (DMSO) δ: 1.27 (m, 2H, CH₂(c)), 1.52 (m, 4H, 2×CH₂ (b and d), 2.19 (t, 2H, CH₂ (a), J=6.3 Hz), 2.53 (t, 2H, CH₂ (e), J=7.8 Hz and J=7.5 Hz), 4.84 (s, 2H, PhCH₂), 7.18 (d, 2H, H arom (x), J=8.2 Hz), 7.24 (d, 2H, H arom (y), J=8.2 Hz), 8.35 (s, 1H, H6), 11.83 (bs, 1H, NH), 11,97 (bs, 1H, COOH). ¹³C NMR (DMSO-d6) δ: 25.17 (CH₂(b)), 29.06 (CH₂(c)), 31.52 (CH₂(d)), 34.44 (CH₂(a)), 35.52 (CH₂(e)), 51.33 (CH₂(Bz)), 95.90 (C—Br), 128.45 (2×CH(y)), 129.41 (2×CH(x)), 134.62 (Cq(e)), 142.76 (Cq), 146.02 (C6), 151.22 (C2), 160.44 (C4), 175.29 (COOH).

HRMS (ESI-TOF) m/z calculated for C₁₇H₁₉N₂O₄⁷⁹Br+Na 417.0426; found 417.0443; calculated for C₁₇H₁₉N₂O₄⁸¹Br+Na 419.0405; found 419.0412.

### 6-[4-(5-Chloro-2,4-dioxo-3,4-dihydro-2H-pyrimidin-1-yl-methyl)-phenyl]-hexanoic acid (59)

¹H NMR (DMSO) δ: 1.28 (m, 2H, CH₂(c)), 1.52 (m, 4H, 2×CH₂ (b and d), 2.21 (t, 2H, CH₂ (a), J=7.3 Hz), 2.55 (t, 2H, CH₂ (e), J=7.8 Hz and J=7.5 Hz), 4.83 (s, 2H, CH₂(Bz)), 7.18 (d, 2H, H arom (x), J=8.1 Hz), 7.24 (d, 2H, H arom (y), J=8.1 Hz), 8.29 (s, 1H, H6), 11.86 (s, 1H, NH), 12.01 (bs, 1H, COOH). ¹³C NMR (DMSO-d6) δ: 25.17 (CH₂(b)), 29.06 (CH₂(c)), 31.53 (CH₂(d)), 34.44 (CH₂(a)), 35.51 (CH₂(e)), 51.37 (CH₂(Bz)), 107.40 (C—Br), 128.44 (2×CH(y)), 129.41 (2×CH(x)), 134.56 (Cq(e)), 142.76 (Cq(Bz)), 143.67 (C6), 150.99 (C2), 160.27 (C4), 175.29 (COOH). HRMS (ESI-TOF) m/z calculated for C₁₇H₁₉N₂O₄³⁵Cl+Na 373.0931; found 373.0921; calculated for C₁₇H₁₉N₂O₄³⁷Cl+Na 375.0902; found 375.0923.

6-[4-(5-Bromo-2,4-dioxo-3,4-dihydro-2H-pyrimidin-1-yl-methyl)-phenyl]-hexanoic acid amide (61) ¹H NMR (DMSO) δ: 1.25 (m, 2H, CH₂(c)), 1.51 (m, 4H, 2×CH₂ (b and d), 2.04 (t, 2H, CH₂ (a), J=7.4 Hz), 2.54 (t, 2H, CH₂ (e), J=7.8 Hz and J=7.5 Hz), 4.84 (s, 2H, CH₂(Bz)), 6.67 and 7.19 (d, 2H, CONH₂, 7.18 (d, 2H, H arom (x), J=8.1 Hz), 7.23 (d, 2H, H arom (y), J=8.1 Hz), 8.35 (s, 1H, H6), 11.82 (s, 1H, NH). ¹³C NMR (DMSO-d6) δ: 25.77 (CH₂(b)), 29.19 (CH₂(c)), 31.59 (CH₂(d)), 35.55 (CH₂(e)), 35.89 (CH₂(a)), 51.33 (CH₂(Bz)), 95.92 (C—Br), 128.44 (2×CH(y)), 129.41 (2×CH(x)), 134.62 (Cq(e)), 142.81 (Cq(Bz)), 146.00 (C6), 151.24 (C2), 160.47 (C4), 175.09 (CONH₂). HRMS (ESI-TOF) m/z calculated for C₁₇H₂₀N₃O₃⁷⁹Br+Na 416.0586; found 416.0571; calculated for C₁₇H₂₀N₃O₃⁸¹Br+Na 418.0565; found 418.0555.

### 6-[4-(5-Chloro-2,4-dioxo-3,4-dihydro-2H-pyrimidin-1-yl-methyl)-phenyl]-hexanoic acid amide (62)

¹H NMR (DMSO) δ: 1.25 (m, 2H, CH₂(c)), 1.51 (m, 4H, 2×CH₂ (b and d), 2.02 (t, 2H, CH₂ (a), J=7.4 Hz), 2.54 (t, 2H, CH₂ (e), J=7.7 Hz and J=7.5 Hz), 4.83 (s, 2H, CH₂(Bz)), 6.67 and 7.19 (each bs, 2H, CONH₂), 7.18 (d, 2H, H arom., J=8.2 Hz), 7.24 (d, 2H, H arom., J=8.2 Hz), 8.29 (s, 1H, H6), 11.86 (s, 1H, NH). ¹³C NMR (DMSO-d6) δ: 25.77 (CH₂(b)), 29.19 (CH₂(c)), 31.59 (CH₂(d)), 34.55 (CH₂(e)), 35.89 (CH₂(a)), 51.38 (PhCH₂), 107.40 (C5), 128.43 (2×CH), 129.41 (2×CH), 134.54 (Cq), 142.81 (Cq), 143.67 (C6), 150.99 (C2), 160.28 (C4), 175.09 (CONH₂). HRMS (ESI-TOF) m/z calculated for C₁₇H₂₀N₃O₃³⁵Cl+Na 372.1091; found 372.1093; calculated for C₁₇H₂₀N₃O₃³⁷Cl+Na 374.1061; found 374.1073.

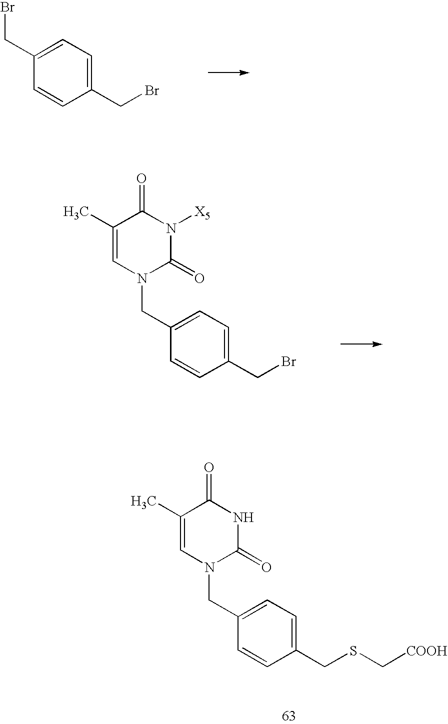

3-[4-(5-Methyl-2,4-dioxo-3,4-dihydro-2H-pyrimidin-1-ylmethyl)-benzylsulfanyl]-acetic acid (63). ¹³C NMR (DMSO-d6) S: 12.79 (CH₃), 23.35 (CH₂), 33.55 (CH₂), 50.60 (PhCH₂), 109.89 (C5), 128.38 and 130.08 (CH arom.), 136.63 and 138.08 (C arom.), 142.12 (C6), 151.86 (C2), 165.10 (C4), 172.101 (COOH). HRMS (ESI-TOF) m/z calculated for C₁₅H₁₆N₂O₄S+Na 343.0728; found 343.0728.

### 3-Fluoro-4-[4-(5-methyl-2,4-dioxo-3,4-dihydro-2H-pyrimidin-1-yl-methyl)-phenyl]-butyraldehyde (64)

HRMS (ESI-TOF) m/z calculated for C₁₆H₁₇N₂O₃F+Na 327.1121; found 327.1098.

### 3-Fluoro-4-[4-(5-Methyl-2,4-dioxo-3,4-dihydro-2H-pyrimidin-1-yl-methyl)-phenyl]-butyric acid (65)

HRMS (ESI-TOF) m/z calculated for C₁₆H₁₇N₂O₄F+Na 343.1070; found 343.1052.

### B—Biological Activity

Activity was determined using the coupled spectrophotometric assay described in Blondin et al. (C. Blondin, L. Serina, L. Wiesmüller, A. -M. Gilles, O. Bârzu, Anal. Biochem. 1994, 220, 219).

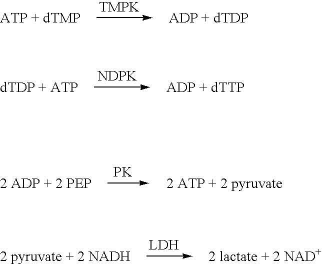

Each mole of transferred phosphoryl group generates two moles of NAD+ and the decrease in absorbance at 334 nm is followed in an Eppendorf ECOM 6122 photometer. The reaction medium (0.5 ml final volume) contained 50 mM Tris-HCl pH 7.4, 50 mM KCl, 2 mM MgCl₂, 0.2 mM NADH, 1 mM phosphoenol pyruvate and 2 units each of lactate dehydrogenase, pyruvate kinase and nucleoside diphosphate kinase. One unit of enzyme activity corresponds to 1 μmole of the product formed in 1 min. at 30° C. and pH 7.4. The concentrations of ATP and dTMP were kept constant at 0.5 mM and 0.05 mM respectively, whereas the concentrations of analogues varied between 0.005 and 8 mM. Equation 1 was used to calculate the Ki values using Equations 2 and 3 (classical competitive inhibition model following the Lineweaver-Burk representation):

$$\begin{matrix}{K}_{i} = \frac{{K}_{m} [ I ]}{( \frac{v}{{v}_{i}} - 1 ) ( {K}_{m} + [ S ] )} & ( Eq . 1 ) \\ v = \frac{{V}_{m} [ S ]}{[ S ] + {K}_{m}} & ( Eq . 2 ) \\ {v}_{i} = \frac{{V}_{m} [ S ]}{[ S ] + {K}_{m} ( 1 + \frac{[ I ]}{{K}_{i}} )} & ( Eq . 3 )\end{matrix}$$

where v and vᵢ are the reaction velocities respectively in the absence and in the presence of the analogue at a concentration value [I]; Km is the Km for dTMP (4.5 μM for TMPKmt and 5 μM for TMPKh); [S] is the concentration of dTMP (50 μM).

The results are exposed in Table 1 and Table 2:

TABLE 1
Biological activity of the molecules 1 to 22.

| | TMPKmt W1002 |
|---|---|
| Compounds | Ki (μM) |
| dTMP | Km = 4.5 |
| dT | 27 |
| 1 | 110 |
| 2 | 68 |
| 3 | 75 |
| 4 | 44 |
| 5 | N.A. |
| 6 | 45 |
| 7 | 90 |
| 8 | 67 |
| 9 | 50 |
| 10 | 44 |
| 11 | 38 |
| 12 | 980 |
| 13 | 810 |
| 14 | 240 |
| 15 | N.A. |
| 16 | N.A. |
| 17 | N.A. |
| 18 | N.A. |
| 19 | 265 |
| 20 | 16.5 |
| 21 | 12.3 |
| 22 | 32 |

TABLE 2
Biological activity of the molecules 28, 30, 31, 39,
40a, 40b, 42, 43, 45, 48, 51 to 54, 58, 59, 61 to 65.

| Compound | Ki (μM) |
|---|---|
| 28 | 48 |
| 30 | 86 |
| 31 | 63 |
| 39 | 15 |
| 40a | 70 |
| 40b | 139 |
| 42 | 48 |
| 43 | 48 |
| 45 | 72 |
| 48 | 47 |
| 51 | 119 |
| 52 | N.A. |
| 53 | 113 |
| 54 | 33 |
| 58 | 42 |
| 59 | 49 |
| 61 | 24 |
| 62 | 32 |
| 63 | 15 |
| 64 | 26 |
| 65 | 63 |

The IC50 value for three compounds has been measured on cultures of Mycobacterium tuberculosis H37Ra. The results are summarized in Table 3:

TABLE 3
Biological activity (IC50) of molecules 20, 21 and 22.

| Compound | IC50 (μg/ml) |
|---|---|
| 20 | 100 |
| 21 | 50 |
| 22 | 25 |

Cytotoxicity: The molecule 22 has been tested for its cytotoxicity on VERO cells. It has been determined that no growth inhibition, and consequently no cytotoxic activity, could be detected up to a concentration of 250 μg/ml of compound 22.

1: A compound of formula (I): 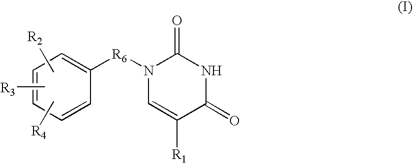
wherein
R₁ is selected from the group consisting of: CH₃, —CF₃, a halogen atom, —NH₂, —COOH, —CONH₂,
R₂, R₃, R₄, identical or different, are selected from the group consisting of:
H, a halogen atom,
C₁-C8 alkyl, C2-C alkenyl, C₂-C₈ alkyl, wherein the alkyl, alkenyl or alkynyl chain may be interrupted by a heteroatom bridge,
—OH, —NH, —CH O, —COOH, —SO₄H, —CONH₂, —CN, —COOR₅, —COR₅, —OR₅,
substituted C₁-C₈ alkyl, substituted C₂-C₈ alkenyl, or substituted C₂-C₈ alkynyl wherein the substituent is selected from the group consisting of: —OH, —NH₂, —CHO, —CO(H, —SO₄H, —CONH₂, —CN, —COOR₅, —COR₅, —OR—, a halogen atom, wherein the alkyl, alkenyl or alkynyl chain may be interrupted by a heteroatom bridge;
R₅ is selected from the group consisting of C₁-C₆ alkyl;
R₆ is selected among: C₁-C₄ alkylene, C₂-C₄ alkenylene, carbonyl (═Cr═O), —(CF₂)ₙ—
n is an integer selected from 1, 2, 3,
and their pharmaceutically acceptable salts,
with the exception of the following cases:
R₁=—CH₃, R₂═R₃═R₄═H and R₆=—CH₂—
R₁=—CF₃, R₂═R₃═R₄═H and R₆=—CH₂—
R₁=—CH₃, R₂═R₃═H, R₄═—OCH₃ (para) and R₆=—CH₂—
R₁=—CH₃, R₂═R₃═CH₃ (ortho, ara, ═H and R₆=—CH—
R₁=—CH₃, R₂═CH₃ (ortho), R₃═R═H and R₆=—CH₂—
R₁=—CH₁, R₂═R₃═R₄═H and R₆═—CO—
R₁═CH₃, R₂═OH (meta), R₃═R₄═H and =—CH₂—
R₂═R₃══H, R₆=—CH₂— and R₁═Cl, I or Br.

2: The compound according to claim 1, wherein one or more of the following conditions is satisfied:
R₆ is —CH₂—.
R₁ is selected or the group consisting Q: —CH₃, —Br, —C1;
at least one group among R₂, R₃, is H.

3: The compound according to claim 1, wherein R₂═R₃═H, R₄ is in the para position on the phenyl ring and is selected from the group consisting of substituted C₁-C₆ alkyl or substituted C₂-C₆ alkenyl, wherein the substituent is —COOH possibly comprising a heteroatom bridge, said heteroatom being selected from: N, S, O, Se.

4: The compound according to claim 1, wherein the compound is selected from the group consisting of:

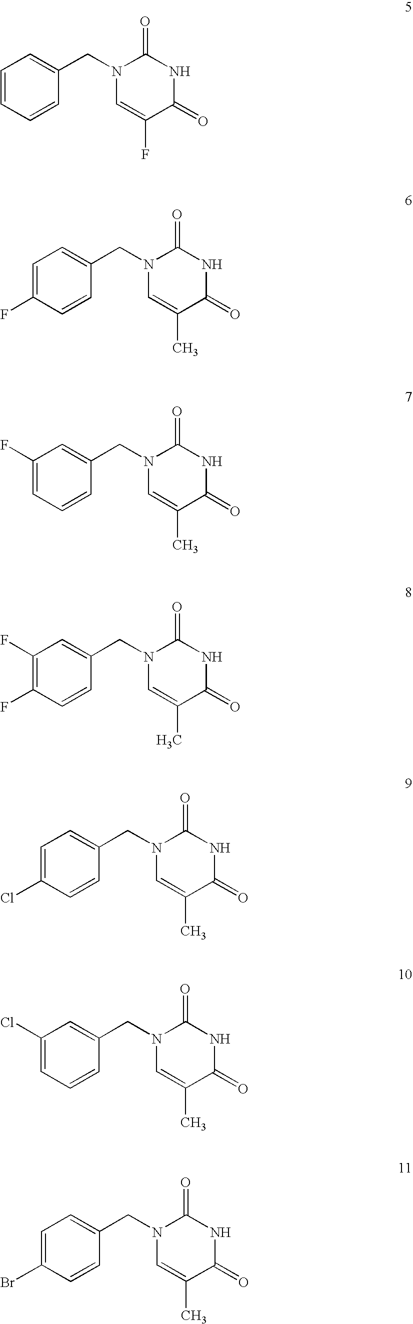

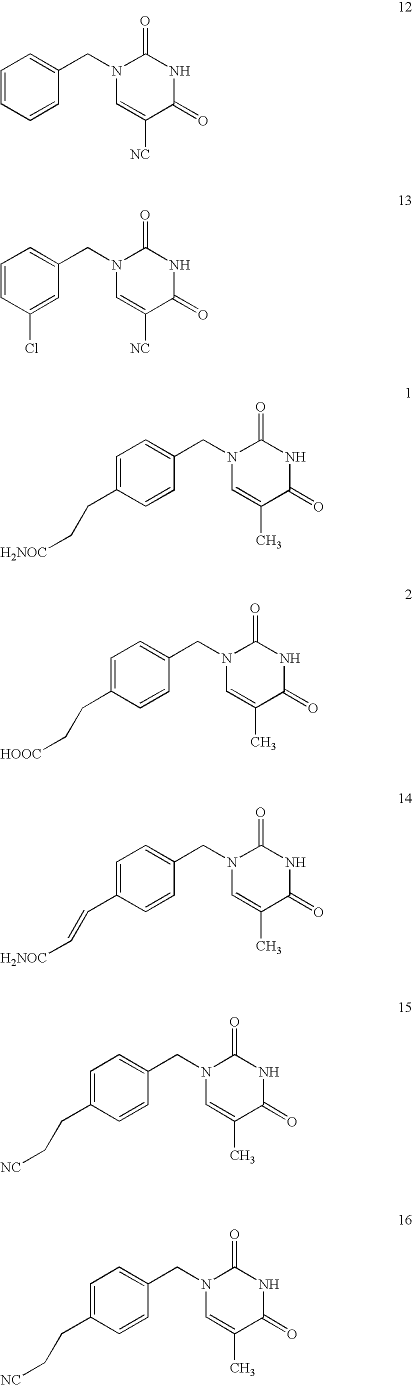

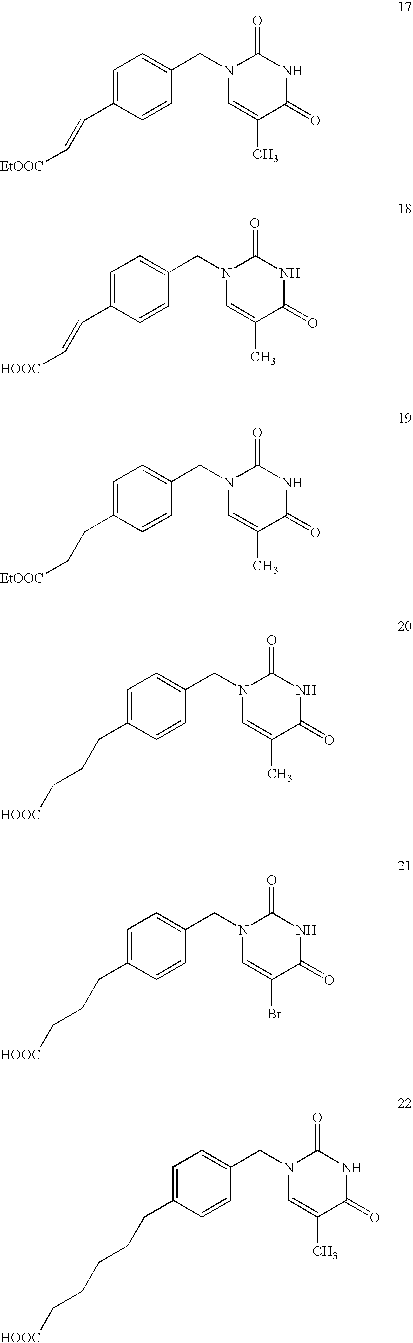

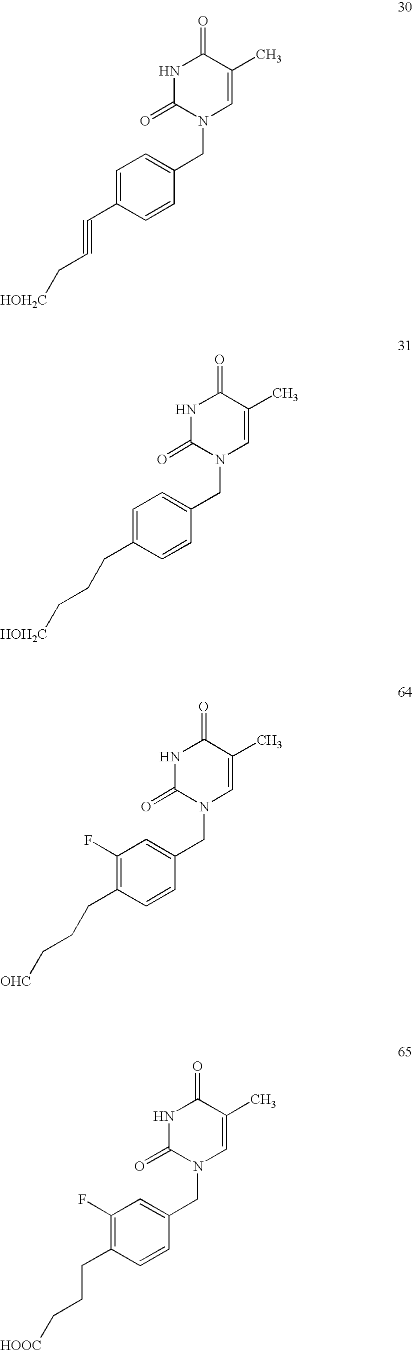

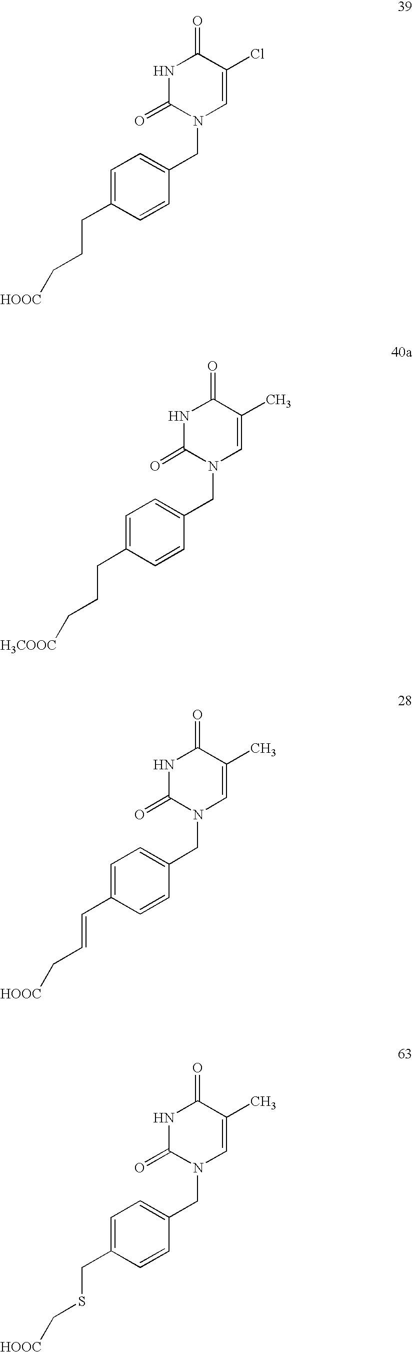

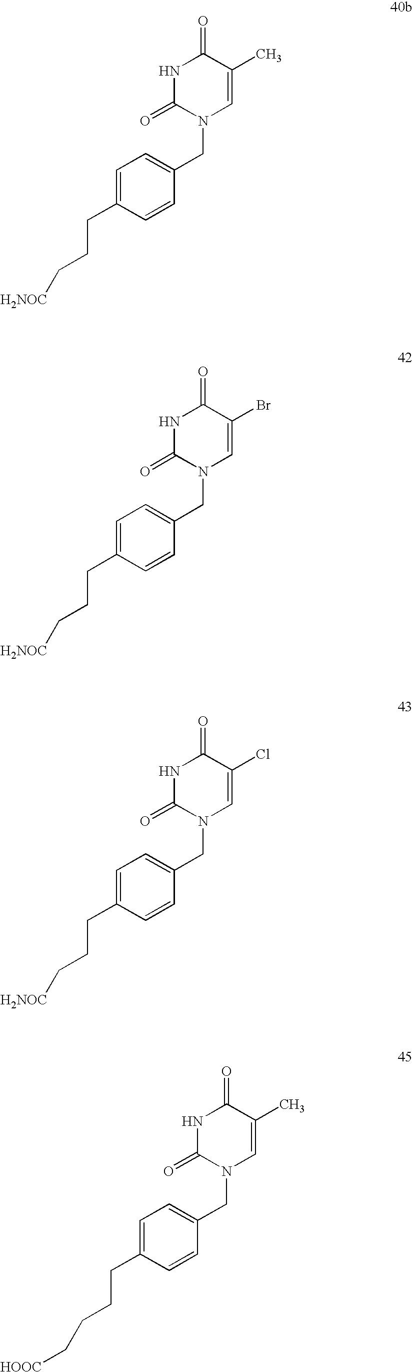

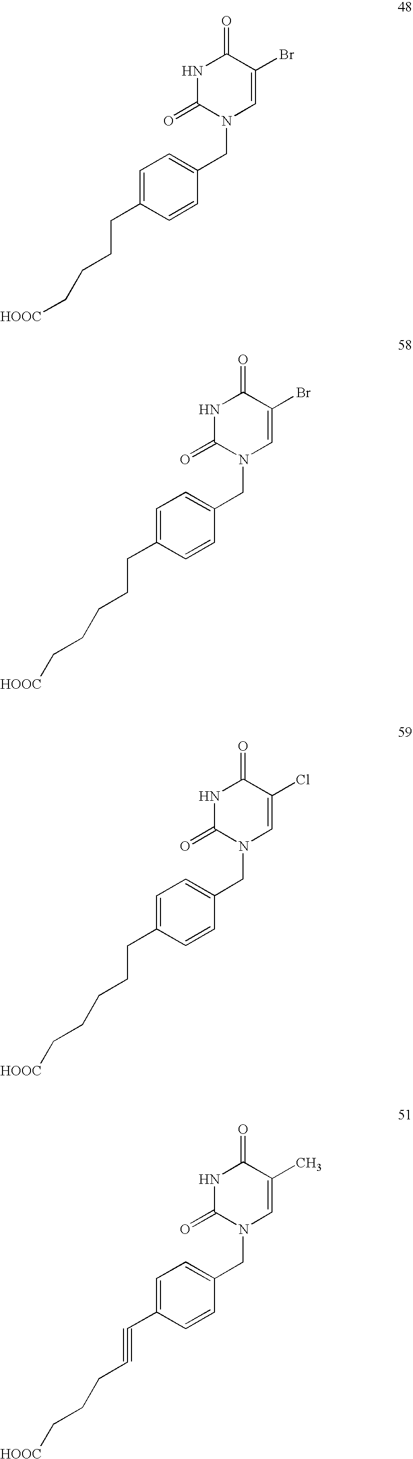

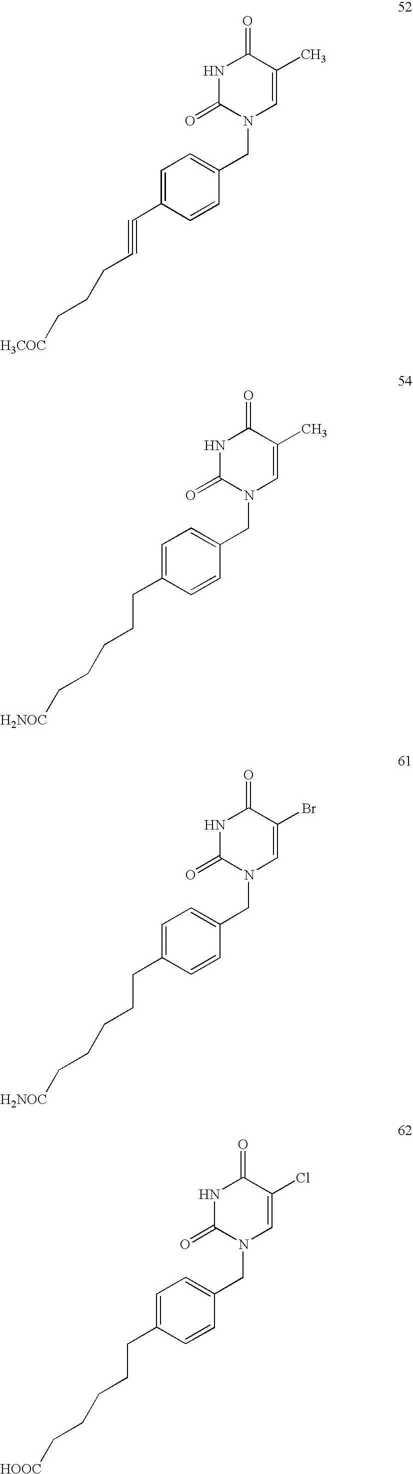

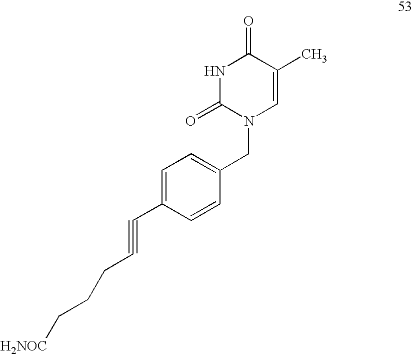

5: The compound according to claim 1, wherein the compound is selected from the group consisting of:

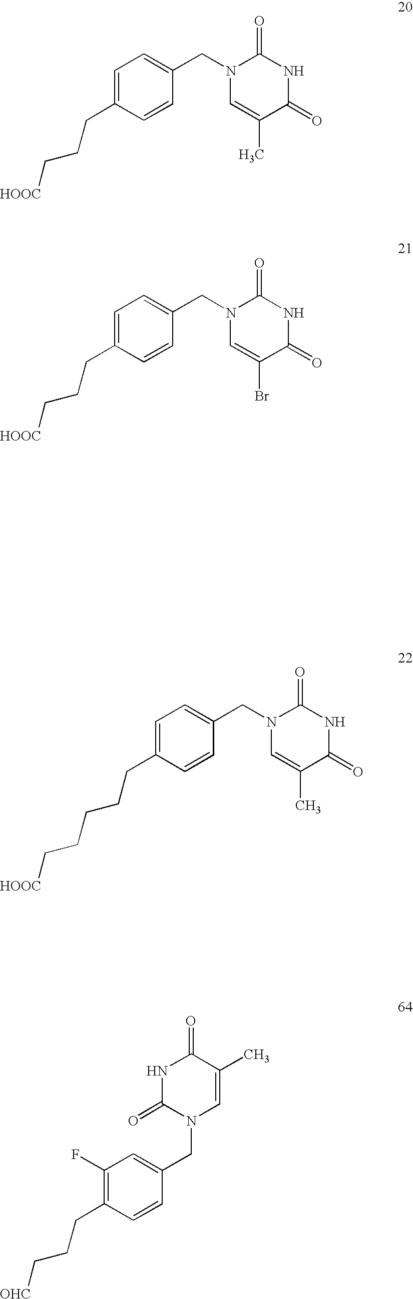

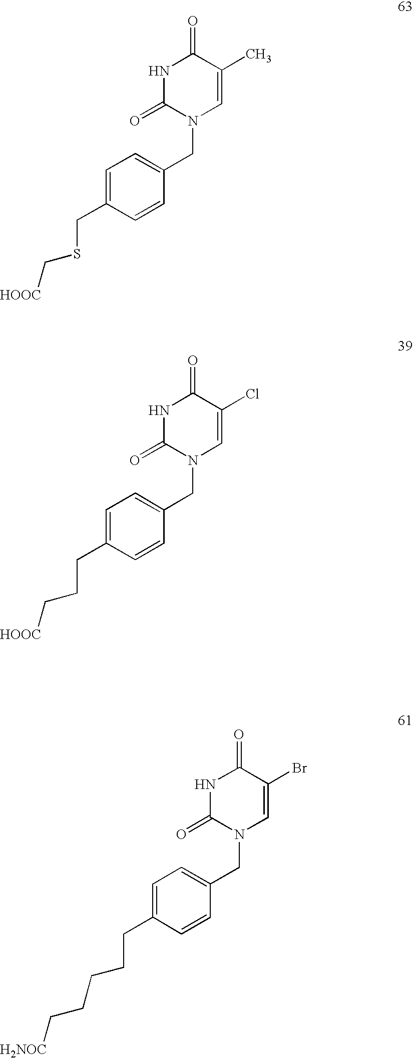

6: A pharmaceutical composition comprising at least one compound of formula (I): 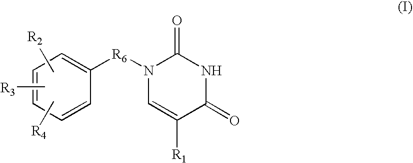
wherein:
R₁ is selected from the group consisting of: CH₃, —CF₃, a halogen atom, —NH₂, —COOH, —CONH₂,
R₂, R₃, identical or different, are selected from the group consisting of:
H, a halogen atom,
C₁-C₈ alkyl, C₂-C₈ alkenyl, C₂-C₈ alkynyl, wherein the alkyl, alkenyl or alkynyl chain may be interrupted by a heteroatom bridge,
—OH, —H₂, —CHO, —COOH, —S₄H, —CONH₂, —CN, —COOR₅, —COR₅, —OR₅,
substituted C₁-C₈ alkyl, substituted C₂-C₈ alkenyl, or substituted C2-C alkynyl wherein the substituent is selected from the group consisting of: —OH, —NH₂, —CHO, —COOH, —SO₄H, —CO 2, —CN, —COOR₅, —COR₅, —OR₅, a halogen atom, wherein the alkyl, alkenyl or alkynyl chain may be interrupted by a heteroatom bridge;
R₅ is selected from the group consisting of C₁-C₆ alkyl;
R₆ is selected among: C₁-C₄ alkylene, C₂-C₄ alkenylene, carbonyl (═C═O) —(CF₂)ₙ—
n is an integer selected on 1, 2, 3,
and their pharmaceutically acceptable salts, in a pharmaceutically acceptable carrier.

7: The pharmaceutical composition comprising at least one compound of formula (I) according to claim 1 and a pharmaceutically acceptable carrier.

8-14. (canceled)

15: A process for the preparation of the compound of formula (I) according to claim 1, comprising:
reacting a haloaryl of formula (II) with a thymine or thymine derivative or uracyle or uracyle derivative of formula (III) to give condensate (IV),
wherein X represents a halogen atom,
X₂, X₃, X₄ are selected among R₂, R₃ and R₄, respectively and a functional group which can be transformed in one or more steps into R₂, R₃ and R₄,
X₁ is selected among R₁ and a functional group which can be transformed in one or more steps into R₁,
X₅ is selected among H and the benzyl group (Bzl); and
transforming, if necessary, X₁, X₂, X₃, X₄ and X₅ into R₁, R₂, R₃, R₄ and H, respectively to give the molecule of formula (I)

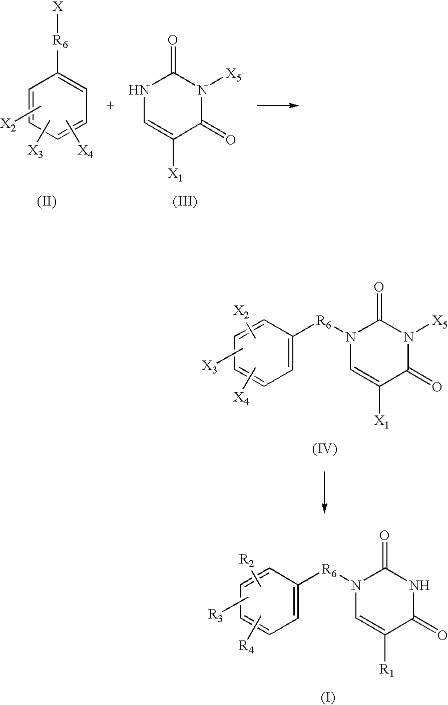

16: A compound of formula (V): 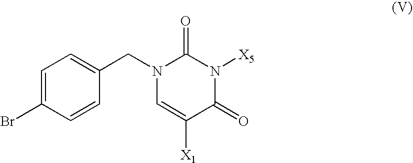
wherein X₁ is selected among R₁ and a functional group which can be transformed in one or more steps into R₁,
R₁ is selected from the group consisting of: CH₃, —CF₃, a halogen atom, —NH₂, —COOH, —CONH₂.
and
X₅ is selected among H and the benzyl group (Bzl).

17: The compound according to claim 16, wherein the compound is selected from the compound of formula 11 and formula 11bis:

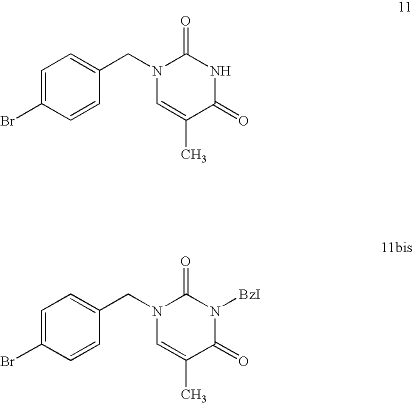

18: The process of claim 15, wherein the halogen atom is Br.

19: A method of treating of tuberculosis and/or leprosy comprising administering to a subject in need thereof an effective amount of a composition comprising as an active ingredient the compound of formula (I):

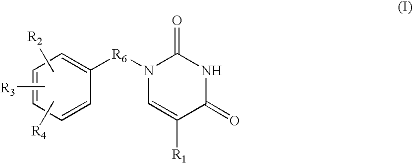

20: The method of claim 18, wherein a daily dose of the of the active ingredient is between 0.1 and 500 mg/kg.

21. The method of claim 18, wherein the compound is an inhibitor of a mycobacteria thymidine monophosphate kinase (TMPK).

22. The method of claim 18, wherein the mycobacteria is M. tuberculosis.
